(19) United States
(12) Patent Application Publication
(10) Pub. No.: US 2016/0128965 A1
(43) Pub. Date: May 12, 2016

(54) GEL COMPOSITIONS

(71) Applicant: LEO Laboratories Limited, Dublin (IE)

(72) Inventors: Per-Ola Arvidsson, Ballerup (DK); Edit Farkas, Ballerup (DK); Chinar Sami Saeed, Ballerup (DK); Mikkel Krogh-Madsen, Ballerup (DK); Andre Huss Eriksson, Ballerup (DK); Kresten Skak, Ballerup (DK); Karsten Petersson, Ballerup (DK)

(21) Appl. No.: 14/738,784

(22) Filed: Jun. 12, 2015

Related U.S. Application Data
(63) Continuation of application No. PCT/EP2013/076188, filed on Dec. 11, 2013.

(30) Foreign Application Priority Data
Dec. 12, 2012 (GB) GB1222407.7
Dec. 6, 2013 (EP) 13171722.5

(51) Int. Cl. A61K 31/22; A61K 9/00; A61K 9/107; A61K 9/06

(52) U.S. Cl. CPC A61K 31/22; A61K 9/06; A61K 9/0014; A61K 9/1075

(57) ABSTRACT

Novel aqueous topical gel compositions comprising ingenol-3-angelate.

### TECHNICAL FIELD

The invention relates to a topical gel composition comprising ingenol-3-angelate as a pharmacologically active agent.

### BACKGROUND OF THE INVENTION

PICATO® is an aqueous gel formulation comprising ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester, also known as ingenol-3-mebutate or PEP005) at a strength of 0.015% or 0.05%. PICATO® was granted regulatory approval in 2012 by the FDA for the topical treatment of actinic keratosis.

The compound ingenol-3-angelate (PEP005) [Sayed, M. D. et. al.; Experienta, (1980), 36, 1206-1207] can be isolated from various Euphorbia species, and particularly from Euphorbia peplos [Hohmann, J. et. al; Planta Med., (2000), 66, 291-294] and Euphorbia drummondii by extraction followed by chromatography as described in U.S. Pat. No. 7,449,492.

Pharmaceutical formulation of the compound has been described in WO2007/068963, which discloses various gel formulations for the treatment of skin cancer.

Angelic acid and angelic acid esters such as ingenol-3-angelate are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F et. al., Chem. Ber. (1970), 103, 561-563].

Furthermore, ingenol-3-acylates are known to be unstable as they rearrange to afford the ingenol-5-acylates and ingenol-20-acylates [Sorg, B. et. al, Z. Natuiforsch., (1982), 37B, 748-756].

It is an object of the invention to provide a composition of ingenol-3-angelate which is stable at room temperature for extended periods.

It is a further object of the invention to provide a composition exhibiting favourable penetration characteristics and biological activity.

It is a further object of the invention to provide a composition with reduced skin irritation, favourable cosmetic properties and improved patient compliance.

A crystalline form of ingenol-3-angelate has been described in WO2011/128780. It is also an object of the invention to utilise the properties of the crystalline structure.

### STATEMENTS OF INVENTION

In one aspect, the present invention relates to an aqueous topical gel composition comprising ingenol-3-angelate, wherein the composition does not consist essentially of:
(i) ingenol-3-angelate, isopropyl alcohol, hydroxyethyl cellulose, citrate buffer and benzyl alcohol; or
(ii) ingenol-3-angelate, a sulfobutyl ether derivative of β-cyclodextrin having from six to seven sulfobutyl ether groups per cyclodextrin molecule (Captisol®) and phosphate buffer; or
(iii) ingenol-3-angelate, dimethyl sulfoxide and phosphate buffer; or
(iv) ingenol-3-angelate, glycerol, cyclomethicone, isopropyl alcohol, carbomer-934, propyl alcohol, water and ethanolamine; or
(v) ingenol-3-angelate, benzyl alcohol, isopropyl alcohol, citrate buffer and hydroxyethyl cellulose; or
(vi) ingenol-3-angelate, benzyl alcohol, citrate buffer, poloxamer 407; or
(vii) ingenol-3-angelate, benzyl alcohol, citrate buffer, poloxamer 407 and propylene glycol; or
(viii) ingenol-3-angelate, benzyl alcohol, citrate buffer and PEG 400.

In another aspect, the present invention relates to an aqueous topical gel composition comprising ingenol-3-angelate, wherein the ingenol-3-angelate is not present in the composition in an amount of 0.015%, 0.02%, 0.05%, 0.08%, 0.09% or 0.1% by weight of the composition.

In another aspect, the present invention relates to an aqueous topical gel composition comprising ingenol-3-angelate as a suspension. In some embodiments, the composition may include ingenol-3-angelate as a non-crystalline suspension. In some embodiments, the composition may include ingenol-3-angelate as a crystalline suspension. The total ingenol-3-angelate in the composition is usefully present at 0.015% by weight or 0.05% by weight of the composition. These suspensions include solid ingenol-3-angelate within the gel, but may also include dissolved ingenol-3-angelate.

In another aspect, the present invention relates to an aqueous topical gel composition comprising ingenol-3-angelate in an amount of from 0.021% to 0.079% by weight of the composition, wherein:
(i) if the ingenol-3-angelate is present in the composition in an amount of 0.05% by weight of the composition, then the composition does not consist essentially of ingenol-3-angelate, isopropyl alcohol, hydroxyethyl cellulose, benzyl alcohol and citrate buffer.

In another aspect, the present invention relates to an aqueous topical gel composition comprising ingenol-3-angelate in an amount of from 0.021% to 0.079% by weight of the composition, wherein:
(i) if the ingenol-3-angelate is present in the composition in an amount of 0.05% by weight of the composition, then the composition does not consist essentially of ingenol-3-angelate, isopropyl alcohol in an amount of 30% by weight of the composition, hydroxyethyl cellulose in an amount of 1.5% of the composition, benzyl alcohol in an amount of 0.9% by weight of the composition and citrate buffer in an amount of 67.55% by weight of the composition.

In another aspect, the present invention relates to an aqueous topical gel composition comprising ingenol-3-angelate in an amount of from 0.001% to 0.019% by weight of the composition, wherein:
(i) if the ingenol-3-angelate is present in the composition in an amount of 0.015% by weight of the composition, then the composition does not consist essentially of ingenol-3-angelate, isopropyl alcohol, hydroxyethyl cellulose, benzyl alcohol and citrate buffer.

In another aspect, the present invention relates to an aqueous topical gel composition comprising ingenol-3-angelate in an amount of from 0.001% to 0.019% by weight of the composition, wherein:
(i) if the ingenol-3-angelate is present in the composition in an amount of 0.015% by weight of the composition, then the composition does not consist essentially of ingenol-3-angelate, isopropyl alcohol in an amount of 30% by weight of the composition, hydroxyethyl cellulose in an amount of 1.5% of the composition, benzyl alcohol in an amount of 0.90% by weight of the composition and citrate buffer in an amount of 67.585% by weight of the composition.

In another aspect, the present invention relates to an aqueous topical gel composition comprising ingenol-3-angelate, wherein:
(i) the ingenol-3-angelate is not present in the composition in an amount of 0.02% by weight of the composition, or from 0.08% to 0.1% by weight of the composition; and
(ii) if the ingenol-3-angelate is present in the composition in an amount of 0.05% or 0.015% by weight of the composition, then the composition does not consist essentially of ingenol-3-angelate, isopropyl alcohol, hydroxyethyl cellulose, benzyl alcohol and citrate buffer.

In another aspect, the present invention relates to an aqueous topical gel composition comprising ingenol-3-angelate, wherein:
(i) the ingenol-3-angelate is not present in the composition in an amount of 0.02% by weight of the composition, or from 0.08% to 0.1% by weight of the composition; and
(ii) if the ingenol-3-angelate is present in the composition in an amount of 0.05% by weight of the composition, then the composition does not consist essentially of ingenol-3-angelate, isopropyl alcohol in an amount of 30% by weight of the composition, hydroxyethyl cellulose in an amount of 1.5% of the composition, benzyl alcohol in an amount of 0.9% by weight of the composition and citrate buffer in an amount of 67.55% by weight of the composition; and
(iii) if the ingenol-3-angelate is present in the composition in an amount of 0.015% by weight of the composition, then the composition does not consist essentially of ingenol-3-angelate, isopropyl alcohol in an amount of 30% by weight of the composition, hydroxyethyl cellulose in an amount of 1.5% of the composition, benzyl alcohol in an amount of 0.90% by weight of the composition and citrate buffer in an amount of 67.585% by weight of the composition.

In another aspect, the present invention relates to an aqueous topical gel composition comprising ingenol-3-angelate, wherein:
(i) the ingenol-3-angelate is not present in the composition in an amount of 0.02% by weight of the composition, or from 0.08% to 0.1% by weight of the composition; and
(ii) if the composition consists essentially of ingenol-3-angelate in an amount of 0.05% by weight of the composition, isopropyl alcohol in an amount of 30% by weight of the composition, hydroxyethyl cellulose in an amount of 1.5% of the composition, benzyl alcohol in an amount of 0.9% by weight of the composition and citrate buffer in an amount of 67.55% by weight of the composition, then the pH of the citrate buffer is other than 2.8; and
(iii) if the composition consists essentially of ingenol-3-angelate in an amount of 0.015% by weight of the composition, isopropyl alcohol in an amount of 30% by weight of the composition, hydroxyethyl cellulose in an amount of 1.5% of the composition, benzyl alcohol in an amount of 0.90% by weight of the composition and citrate buffer in an amount of 67.585% by weight of the composition, then the pH of the citrate buffer is other than 2.8.

In another aspect, the present invention relates to an aqueous topical gel composition comprising ingenol-3-angelate, wherein the composition comprises one or more silicones and does not consist essentially of ingenol-3-angelate, glycerol, cyclomethicone, isopropyl alcohol, carbomer-934, propyl alcohol, water and ethanolamine.

In another aspect, the present invention relates to an aqueous topical gel composition comprising ingenol-3-angelate, wherein the composition comprises one or more silicones and does not consist essentially of ingenol-3-angelate in an amount of 0.02% by weight of the composition, glycerol, cyclomethicone, isopropyl alcohol, carbomer-934, propyl alcohol, water and ethanolamine.

In another aspect, the present invention relates to an aqueous topical gel composition comprising ingenol-3-angelate, wherein the composition comprises one or more silicones and the pH is less than about 4.5.

In another aspect, the present invention relates to an aqueous topical gel composition comprising ingenol-3-angelate, wherein the ingenol-3-angelate is present in the composition in an amount of from 0.001% to 0.019% by weight of the composition and the composition comprises one or more silicones.

In another aspect, the present invention relates to an aqueous topical gel composition comprising ingenol-3-angelate, wherein the ingenol-3-angelate is present in the composition in an amount of from 0.021% to 0.089% by weight of the composition and the composition comprises one or more silicones.

In another aspect, the present invention relates to an aqueous topical gel composition comprising ingenol-3-angelate, wherein the composition comprises one or more silicones and does not comprise ethylamine.

In another aspect, the present invention relates to an aqueous topical gel composition comprising ingenol-3-angelate, wherein the composition is not a water-in-oil emulsion including an oily phase comprising:
(a) ingenol-3-angelate in dissolved form;
(b) at least one non-ionic surfactant selected from the group consisting of polyoxyl glycerides, polyoxyethylene castor oil derivatives, polyoxyethylene alkyl ethers, polysorbates, or a mixture of acrylamide acryloyldimethyl taurate copolymer, isohexadecane, polysorbate 80, sterols, fatty alcohols, fatty acid phosphonates, mono- or diglycol esters, mono-di- or polyglyceryl esters, mono-, di- or polyglucose esters, sucrose esters or sorbitan esters, the non-ionic surfactant being present in an amount of from about 0.5% to about 10% by weight of the composition;
(c) a solvent for the ingenol-3-angelate; and
(d) an aqueous phase buffered to a pH of 2.6-3.7.

The present invention further relates to methods for treating a dermal disease or condition comprising topical administration of a gel of the invention.

### BRIEF DESCRIPTION OF DRAWINGS

FIG. 1 shows the percentage of applied ingenol-3-angelate which penetrates into the viable epidermis and dermis (dark shading) and the percentage of applied ingenol-3-angelate which permeates to receptor fluid (light shading) according to the in vitro diffusion test for composition 33, formulations 02A and 09A, and PICATO® at the same strength of ingenol-3-angelate by weight of the composition.

FIG. 2 shows the amount of ingenol-3-angelate in the skin (penetrating into the viable epidermis and dermis)) according to the in vitro diffusion test for composition 73-07A, 73-08A and 73-10A.

FIG. 3 shows the effect in the B-16 melanoma mice model. Survival curves of compositions 73-07A, 73-08A and 73-11A are presented.

FIG. 4 shows the composite local skin reaction (LSR) in hairless guinea pigs of composition 68-01A and 68-16A.

### DETAILED DESCRIPTION OF THE INVENTION

The present composition has been found to result in a satisfactory chemical stability of ingenol-3-angelate permitting the composition to be stored at room temperature (25° C.) for extended periods.

Human skin, in particular the outer layer, the stratum corneum, provides an effective barrier against penetration of microbial pathogens and toxic chemicals. While this property of skin is generally beneficial, it complicates the dermal administration of pharmaceuticals in that a substantial amount of an active ingredient applied on the skin of a patient suffering from a dermal disease may not penetrate into the viable layers of the skin (the dermis and epidermis) where it exerts its activity. The gel compositions of the present invention may provide advantageous penetration properties whilst reducing the likelihood of skin irritation.

Gels

Gels are semisolid dosage forms that contain an agent (a gelling agent) to provide stiffness to a solution or a colloidal dispersion. Gels do not flow at low shear stress and generally exhibit plastic flow behaviour. The gel compositions of the present invention could be hydrogels.

Whether or not a system behaves as a gel (i.e. exhibits semisolid characteristics, rather than acting as a liquid or solid, etc.) will depend on the various components used within the system and the relative ratios of the different components. It may also depend on the method by which the components that make up the system are mixed, e.g. the order in which the various components are introduced to each other. It is therefore possible for an agent to act as a gelling agent in one environment but not in another. The ability to test compositions to confirm that they are gels as defined herein is within the knowledge of the skilled person in view of the present disclosure and common general knowledge in the field.

The viscosity of a gel can depend on temperature. At low temperatures (e.g. 2-8° C.) the viscosity can be relatively high, but after applying a gel composition of the invention to the skin it can become less viscous because of the combination of increased temperature and the physical stress while being applied. This shear-thinning characteristic gives a gel which is easily distributed on the skin.

In order to effect formation of a gel, it is necessary to have an agent in the composition which acts as a gelling agent. The amount of the gelling agent (or gelling agents, in embodiments where two or more gelling agents are used) required to form a gel will vary on the components within the particular composition. It is common (although not required) to select two or more components which, when used together in particular amounts, effect formation of a gel. These components would typically include an emulsifier and/or viscosity-increasing ingredient with an aqueous buffer solution.

In some embodiments the gel compositions are colourless. In other embodiments they include a coloured substance, which can make it easy to see where the gel has been applied.

Gel compositions of the invention are usually transparent. In some embodiments, the gel compositions include suspended ingenol-3-angelate solids. In these embodiments, the gel compositions are preferably transparent except for the suspended ingenol-3-angelate solids. In other less preferred embodiments, the gel compositions are turbid in appearance.

The gel compositions of the invention are typically acidic, because it has been found that alkaline conditions (or even insufficiently strong acidic conditions) may contribute to degradation of ingenol-3-angelate within the gel composition. This means that the gel compositions are sufficiently acidic for the ingenol-3-angelate to remain stable at room temperature (25° C.) for extended periods, e.g. for 2 years. Generally, the aqueous compositions of the invention will have a pH of from about 2 to about 6, e.g. pH 2, 2.5, 3, 3.5, 4, or 4.5. Although not required, the compositions of the invention will typically include an aqueous buffer solution. Preferably, the gel compositions have a pH of less than about 4.5, such as less than 4 or less than 3.5.

In general, gels are non-invasive and have a localized effect with minimum side effects. Gel compositions of the invention should be suitable for human topical administration. Thus the compositions have the appropriate physical characteristics of topical gels. For instance, the gel compositions have good spreadability, i.e. the gels can readily be spread (e.g. using fingers) after application to the skin to provide a uniform layer. The gel compositions also have excellent extrudability. These properties mean that the gel compositions of the invention are particularly suitable for topical administration. In some embodiments, the gel compositions are applied topically and do not leave a visible residue. The volatile components of the gel compositions may also substantially evaporate to dryness after a certain period of time following topical application. Preferably, the volatile components of the gel composition will evaporate after a therapeutically effective amount of the ingenol-3-angelate has penetrated into the skin (e.g. after about 1 minute, 2 minutes, 5 minutes, 10 minutes, 20 minutes, etc. following topical administration to a subject).

Ingenol-3-Angelate

The composition of the invention includes ingenol-3-angelate. Typically, the composition includes ingenol-3-angelate in an amount of from about 0.001% to about 0.5% by weight of the composition. The composition may include ingenol-3-angelate in an amount of about 0.0005%, 0.001%, 0.0025%, 0.005%, 0.01%, 0.015%, 0.025%, 0.05%, 0.075%, 0.1%, 0.125%, 0.15%, 0.2%, 0.25% or 0.5% by weight of the composition. In two particularly preferred embodiments the composition includes ingenol-3-angelate in an amount of 0.05% or 0.015% by weight of the composition.

The compositions of the present invention can be manufactured, for example, from micro- or nano-processed solid state ingenol-3-angelate. Such micro- or nano-processed solid state ingenol-3-angelate can be produced using various techniques, such as micronization (e.g. by ball mill grinding), nanoprocessing (e.g. by grinding), high pressure homogenization and microfluidization.

Ingenol angelate exists in three isoforms: ingenol-3-angelate (isoform ‘b’), ingenol-5-angelate (isoform ‘a’) and ingenol-20-angelate (isoform ‘c’). The compositions of the present invention include ingenol-3-angelate, i.e. isoform ‘b’, which tends to undergo rearrangement to isoform ‘a’ and subsequently to isoform ‘c’. Preferably, the composition includes less than about 1%, and even more preferably less than about 0.5%, of the ‘a’ isoform after a period of 3 months at room temperature (25° C.). Preferably, the composition includes less than about 1%, and even more preferably less than about 0.5%, of the ‘c’ isoform after a period of 3 months at room temperature (25° C.).

In some embodiments, the compositions of the invention include suspended ingenol-3-angelate solids. The composition may include crystalline ingenol-3-angelate. The crystalline form is readily soluble and so may be particularly preferred, because manufacturing compositions containing crystalline ingenol-3-angelate may be more time and cost efficient than using amorphous ingenol-3-angelate. In certain embodiments, the compositions of the invention include crystalline ingenol-3-angelate in which the crystalline form is not a solvate. In certain embodiments, the compositions of the invention include crystalline ingenol-3-angelate in which the crystalline form is orthorhombic. In certain embodiments, the compositions of the invention include crystalline ingenol-3-angelate in which the crystalline form is characterized by an FTIR-ATR spectrum exhibiting attenuated total reflectance peaks at approximately 3535, 2951, 1712, 1456, 1378, 1246, 1133, 1028 and/or 956 cm⁻¹ (±3 cm⁻¹). In certain embodiments, the compositions of the invention include crystalline ingenol-3-angelate in which the crystalline form has a differential scanning calorimetry curve comprising an event with an onset at about 153±about 5° C. Preferably, when the compositions of the invention include crystalline ingenol-3-angelate, the ingenol-3-angelate has a polymorphic purity of at least about 80%, such as about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the compositions of the invention include amorphous (non-crystalline) ingenol-3-angelate. The compositions can include a mixture of amorphous and crystalline ingenol-3-angelate.

In embodiments where the gel compositions include suspended ingenol-3-angelate solids, they are suspensions, i.e. homogeneous mixtures containing solid particles. These gel compositions may therefore also be described as particulate gels. For example, the amount of dissolved ingenol-3-angelate may vary from about 1% to about 99% by weight of the total amount of ingenol-3-angelate in the gel. Typically where the gel compositions include suspended ingenol-3-angelate solids, about 20% to about 25% of the total ingenol-3-angelate is dissolved within the gel. In some embodiments, substantially all of the ingenol-3-angelate is dissolved. In some embodiments, the compositions may be supersaturated, including both dissolved and solid ingenol-3-angelate.

Buffers

The aqueous compositions of the invention typically include an aqueous buffer solution. The use of buffer solutions means that fluctuations in pH can be minimised and thus the pH can be kept more readily within the desired pH range, such as at a pH of less than about 4.5. This reduces the tendency of the ingenol-3-angelate to degrade to form the tiglate ester, which typically occurs in more basic conditions.

Suitable buffer solutions that can be used in the compositions of the invention include e.g. citrate buffer, phosphate buffer, acetate buffer and citrate-phosphate buffer. A citrate buffer is particularly preferred. If a buffer solution is used in the compositions, the pH of the composition will depend on the amount of buffer and the pH of the buffer used. Typically, the compositions of the invention comprise from about 2.5% to about 90% buffer solution by weight of the composition, e.g. 2.5%, 5%, 10%, 20%, 30%, 40%, 50%, 60%, 70%, 80% or 90% buffer solution by weight of the composition. The pH of the buffer will typically be between about 2 to about 4.5, e.g. pH 2, 2.5, 3, 3.5, 4, or 4.5. A buffer having a pH of from about 2 to about 3 is particularly preferred, because this pH range may permit the composition to be stored at room temperature (25° C.) for extended periods. For instance, in preferred embodiments the pH of the buffer is 2.0, 2.1, 2.2, 2.3, 2.4, 2.5, 2.6, 2.7, 2.8, 2.9 or 3.0. Most preferably, the buffer will be a citrate buffer having a pH of from about 2 to about 3. For example, a citrate buffer can be made by mixing sodium citrate with water. Methods of making buffers of the type disclosed herein are well known to the skilled person.

Emulsifiers

In some embodiments, the composition may include an emulsifier. The emulsifier can function as a gelling agent, such that e.g. formation of a gel may be effected when an emulsifier is added to a mixture of ingenol-3-angelate and an aqueous buffer solution.

The composition may include one or more emulsifiers selected from e.g. group of hydrophilic and lipophilic surfactants or polymers. In embodiments of the invention the emulsifiers are selected from the following group: polyacrylates, polycarbophils, poloxamers, hyaluronic acid, xanthan, natural polysaccharides, chitosan and cellulose-derivatives. Suitable cellulose-derivative emulsifiers include hydroxyalkyl cellulose polymers (e.g. hydroxymethyl cellulose, hydroxyethyl cellulose, hydroxypropyl cellulose (hypromellose) and hydroxypropylmethyl cellulose), carboxymethyl cellulose, methylhydroxyethyl cellulose and methylcellulose, carbomer (e.g. Carbopol®), carrageenans, Poloxamers such as poloxamer 407 and poloxamer 188, PEG fatty esters such as (PEG150 distearate) or PEG castor oil derivative (macrogolglycerol ricinoleate), PEG fatty alcohol ether (macrogol oleyl ether) and phospholipids (lecithin). Glycerol monocarprylocaprate is an oily solvent, which may also be used as emulsifier.

In certain preferred embodiments, the emulsifier is hydroxyethyl cellulose, such as that available under the trade name Natrosol® (e.g. Natrosol® 250 HX, Natrosol® PLUS CS, Grade 300 etc.) and METHOCEL®. In certain preferred embodiments, the emulsifier is hydroxypropyl cellulose, such as that available under the trade name Klucel® and METHOCEL®. In certain embodiments of the invention the composition includes a hydrophilic non-ionic surfactant and/or a lipophilic non-ionic surfactant.

The term “hydrophilic surfactant” means an oil-in-water surfactant with a hydrophilic-lipophilic balance (HLB) value of 9-18, and “lipophilic surfactant” means a water-in-oil surfactant with an HLB value of 1.5-9. By way of an example, polysorbate 80 has an HLB value of 15 and is therefore a hydrophilic surfactant, whereas sorbitan trioleate has an HLB value of 1.8 and is therefore a lipophilic surfactant. The HLB of mixed surfactants is calculated according to their relative weightings (by volume) e.g. a 1:1 mixture by volume of polysorbate 80 and sorbitan trioleate has a HLB of 8.4.

In one embodiment, the composition includes a hydrophilic non-ionic surfactant in an amount of from about 1% to about 40% by weight of the composition, optionally from about 2% to about 15% by weight of the composition. Preferably, the composition includes a hydrophilic non-ionic surfactant in an amount of from about 2% to about 10% by weight of the composition, such as from about 2.5% to about 5% by weight of the composition.

The hydrophilic non-ionic surfactant may be a polyethylene glycol ester of a vegetable oil containing at least 20 moles of ethylene oxide groups/mole of glyceride. Suitable polyethylene glycol esters are typically selected from polyoxyethylene castor oil derivatives (e.g. PEG 20, 30, 35, 38, 40, 50 and 60 castor oil or PEG 20, 25, 30, 40, 45, 50, 60 and 80 hydrogenated castor oil), PEG 20 and 60 corn glycerides, PEG 20 and 60 almond glycerides, PEG 40 palm kernel oil, sodium laurate sulfate, sucrose esters (e.g. sucrose stearate, sucrose distearate, sucrose cocoate or sucrose monolaurate), PEG cocoglyceride, PEG 8 caprylocaprate, polyglyceryl esters and linolenamide DEA. In a preferred embodiment, the hydrophilic non-ionic surfactant is sucrose distearate, such as that available under the trade name Sisterna® SP30.

In certain embodiments, the hydrophilic non-ionic surfactant may be a mixture of acrylamide acryloyldimethyl taurate copolymer, isohexadecane and polysorbate 80, such as that available under the trade name SEPINEO P600. The hydrophilic non-ionic surfactant may be an alkylpolyglucoside, such as that available under the trade name SEPINEO SE68.

In one embodiment, the composition includes a lipophilic non-ionic surfactant in an amount of from about 0.1% to about 5% by weight of the composition. In other embodiments, the lipophilic non-ionic surfactant may be present in an amount of from about 0.1% to about 40% by weight of the composition. Surfactants are generally irritants, and so it is preferred to use only low levels of certain surfactants. However, some lipophilic non-ionic surfactants, such as monoglyceride esters, are less irritative and so can be present in higher amounts without causing significant levels of skin irritation.

The lipophilic non-ionic surfactant may be selected from monoglyceride esters of C₆₋₂₂ fatty acids (e.g. glyceryl monocaprylate, glyceryl monocaprate, glyceryl monostearate, glyceryl monobehenate), diglyceride esters of C₆₋₂₂ fatty acids (e.g. glyceryl dilaurate), mono- and diglyceride esters of C₆₋₂₂ fatty acids (e.g. caprylic/capric mono- and diglyceride, glyceryl mono- and diricinoleate), propylene glycol esters of C₆₋₂₂ fatty esters (e.g. propylene glycol monocaprylate, propylene glycol monolaurate), dialkylene glycol monoalkyl ethers (e.g. diethylene glycol monoethyl ether), polyglyceryl C₆₋₂₂ fatty acid esters (e.g. polyglyceryl-3-diisostearate), polyethylene glycol esters of a triglyceride/vegetable oil containing 4 to 8 moles of ethylene oxide groups/mole of glyceride (e.g. PEG-6 corn oil, PEG-6 almond oil, PEG-6 apricot kernel oil, PEG-6 olive oil, PEG-6 peanut oil, PEG-6 palm kernel oil, hydrogenated palm kernel oil, PEG-6 triolein, PEG-8 corn oil), polysorbates (e.g. polysorbate 20, polysorbate 40, polysorbate 60, polysorbate 80). In a preferred embodiment, the lipophilic non-ionic surfactant is a sorbitan ester, such as that available under the trade name Span® 120. In a preferred embodiment, the lipophilic non-ionic surfactant is an oleoyl macrogol-6 glyceride, such as that available under the trade name Labrafil® M1944 or a lauroyl polyoxyl-6 glyceride, such as that available under the trade name Labrafil® M2130. In some embodiments the emulsifier includes other than non-ionic surfactants. In other embodiments the surfactant is a phospholipid, such as natural or synthetic phospholipids, saturated or unsaturated phospholipids, or phospholipid-like molecules. The phospholipid typically includes one or more saturated or unsaturated acyl moieties. In some embodiments the unsaturated acyl moiety is C₁₂-C₂₄ alkenyl. In some embodiments the saturated acyl moiety is C₁₂-C₂₄ alkyl.

Particularly suitable phospholipids include e.g. soybean lecithin, egg lecithin, lecithin, lysolecithin, phosphatidylserine, phosphatidylethanolamine, phosphatidylcholine and phosphatidylinositol, phosphatidylglycerol, phosphatidylacid, etc. In some embodiments, the phospholipids are mixed with a sterol such as cholesterol, which can stabilize the phospholipid system. In some embodiments, the lipid surfactant is chemically or physically modified. Modifications alter the properties of the lipid surfactant and of the resulting vesicles. Methods of modifying lipid surfactants are known in the art of vesicular formulations. In preferred embodiments, the gel composition includes a phospholipid such as those available under the trade names Phospholipon 900, Phospholipon 19H, NanoSolve® or Lipoid SPC®.

Typically, the emulsifier is present in an amount of from about 0.1% to about 60% by weight of the composition, such as about 0.1%, 3%, 5%, 10%, 15%,20%, 30%, 40%, 50% or 60% by weight of the composition. In an embodiment the emulsifier is present is present in an amount of from about 1% to about 20% by weight of the composition. The composition may include more than one emulsifier, such as two or three emulsifiers.

Non-Aqueous Carrier

Aqueous compositions of the invention may include a pharmaceutically acceptable non-aqueous carrier. The non-aqueous carrier may function as a vehicle for the ingenol-3-angelate, and the ingenol-3-angelate is typically dispersed throughout the carrier. The compositions of the invention can include more than one non-aqueous carrier, e.g. two, three, four or five non-aqueous carriers. The one or more non-aqueous carriers are typically present in the compositions in a combined amount of from about 1% to about 98% by weight of the composition, e.g. about 70% by weight of the composition.

In some embodiment, the non-aqueous carrier can act as an occlusive agent, e.g. it can form a layer on the surface of the skin on application of the composition. This layer can form a hydration barrier sufficient to result in reduction of trans-epidermal water loss, thereby improving in skin hydration.

The non-aqueous carrier may be selected from a mineral oil (e.g. liquid paraffin) or a hydrocarbon or mixture of hydrocarbons with chain lengths ranging from C₅ to C₆₀. The non-aqueous carrier may be petrolatum or white soft paraffin. Such a mixture is usually composed of hydrocarbons of different chain lengths peaking at about C₄₀₄₄. The non-aqueous carrier may comprise a mixture of petrolatum and liquid paraffin. Such a mixture may consist of hydrocarbons of different chain lengths peaking at C₂₈₋₄₀.

While petrolatum provides occlusion of the treated skin surface, reducing transdermal loss of water and potentiating the therapeutic effect of the active ingredient in the composition, it tends to have a greasy and/or tacky feel which persists for quite some time after application, and it is not easily spreadable on the skin. It may therefore be preferred to employ paraffins consisting of hydrocarbons of a somewhat lower chain length, e.g. paraffins comprising hydrocarbons with chain lengths peaking at C₁₄₋₁₆, C₁₈₋₂₂, C₂₀₋₂₂, C₂₀₋₂₆ or mixtures thereof. The hydrocarbon composition of the paraffins can be determined using gas chromatography. It has been found that paraffins comprising hydrocarbons with chain lengths peaking at C₁₄₋₁₆, C₁₈₋₂₂, C₂₀₋₂₂, C₂₀₋₂₆ or mixtures thereof are more cosmetically acceptable because they are less tacky and/or greasy on application and more easily spreadable. They are therefore expected to result in improved patient compliance. Suitable paraffins of this type, which are generally termed petrolatum jelly, are manufactured by Sonneborn and marketed under the trade name Sonnecone. In preferred embodiments of the invention the non-aqueous carrier is selected from Sonnecone CM, Sonnecone DM1, Sonnecone DM2 and Sonnecone HV. These paraffins are further disclosed and characterized in WO 2008/141078 which is incorporated herein by reference.

In some embodiments the non-aqueous carrier is an iso-paraffin, e.g. isohexadecane or squalane.

The non-aqueous carrier may also be a silicone. However, the present invention excludes the two silicone-containing compositions disclosed in WO2007/068963.

In some embodiments, the silicone is cyclic, e.g. cyclomethicone. In other embodiments, the silicone can be linear. In other embodiments, the silicone may be branched. Silicones such as cyclomethicone and dimethicone may be used to reduce the viscosity of the composition, for example in embodiments which also include a silicone of higher viscosity.

In some embodiments, the silicone is a solid mixture of stearoxytrimethylsilane and stearyl alcohol, such as that available under the trade name Dow Corning® Silky Wax 10. In some embodiments, the silicone is a mixture of high molecular weight silicone elastomer (12%) and decamethylcyclopentasiloxane (i.e. a cyclopentasiloxane and dimethicone crosspolymer), such as that available under the trade name Dow Corning® ST-Elastomer 10. In other embodiments, the silicone is comprised of a volatile polydimethylcyclosiloxane composed mainly of decamethylcyclopentasiloxane, such as that available under the trade name Dow Corning® ST cyclomethicone (5-NF). Dow Corning® ST cyclomethicone (5-NF) is particularly useful when the composition comprises a further silicone of higher viscosity, such as Dow Corning@ ST-Elastomer 10. In some embodiments, the silicone comprises a cyclopentasiloxane and polyoxyethylene/polyoxypropylene dimethicone, such as that available under the trade name Dow Corning@ BY 11-030. In some embodiments the composition includes more than one silicone non-aqueous carrier, e.g. two or three silicones.

The non-aqueous carrier may also be an oily solvent. In one embodiment, the oily solvent may be a C₆₋₂₂ acylglyceride, where C₆₋₂₂ acylglyceride means a triglyceride or a mixture of mono- and diglycerides or mono-, di- and triglycerides of C₆₋₂₂ fatty acids, where C₆₋₂₂ acylglyceride means a triglyceride or a mixture of mono- and diglycerides or mono-, di- and triglycerides of C₆₋₂₂ fatty acids. For example, the oily solvent may be a vegetable oil (e.g. sesame oil, sunflower oil, palm kernel oil, corn oil, safflower oil, olive oil, avocado oil, jojoba oil, almond oil, canola oil, coconut oil, cottonseed oil, peanut oil, soybean oil, wheat germ oil, grape kernel oil etc.), or highly purified vegetable oil (e.g. medium chain triglycerides, long chain triglycerides, castor oil, caprylic/capric mono- and diglycerides, caprylic/capric mono-, di- and triglycerides, etc.). Medium chain triglycerides are triglyceride esters of fatty acids with a chain length of 6-12 carbon atoms. A preferred medium chain triglyceride is a mixture of caprylic (C₈) and capric (C₁₀) triglycerides, e.g. available under the trade name Miglyol 812. Other particularly suitable oily solvents include fatty acid glycerol polyglycol esters, e.g. available under the trade name Cremophor RH40. Particularly suitable caprylic/capric glycerides are available under the trade name Akoline MCM or glycerol monocarprylocaprate.

In another embodiment, the oily solvent may be a synthetic oil such as a fatty alcohol ester of a C₁₀₋₁₈ alkanoic acid (e.g. isopropyl myristate, isopropyl palmitate, isopropyl linoleate, isopropyl monooleate and isostearyl isostearate etc.), such as that available under the trade name Polawax®.

In another embodiment, the oily solvent may be a polyoxypropylene fatty alkyl ether (e.g. polyoxypropylene-15-stearyl ether, polyoxypropylene-11-stearyl ether, polyoxypropylene-14-butyl ether, polyoxypropylene-10-cetyl ether or polyoxypropylene-3-myristyl ether etc.). The oily solvent may be a stearyl ether such as that available under the trade name Arlamol® E.

The oily solvent may be an alkyl or dialkyl ester such as ethyl oleate, diisopropyl adipate or cetearyl octanoate. The oily solvent may also be a mono- or diglyceride such as glyceryl monooleate, or a fatty alcohol such as oleyl alcohol.

In some embodiments the composition may include a mixture of two oily solvents, or optionally three oily solvents.

Viscosity-Increasing Ingredient

The gel compositions may include a viscosity-increasing ingredient. For example, when the composition comprises a substantial amount of aqueous buffer solution (e.g. above about 60% by weight of the composition), it may be necessary to add one or more viscosity-increasing ingredients (e.g. in an amount of e.g. about 5% by weight of the composition) in order to form a gel. The viscosity-increasing ingredient may therefore function as the gelling agent. However, there may be no requirement for the additional of a viscosity-increasing ingredient if other components in the composition are capable of acting as a gelling agent.

The viscosity-increasing ingredient can be a wax. The wax may be a mineral wax composed of a mixture of high molecular weight hydrocarbons (e.g. saturated C₃₅₋₇₀ alkanes), such as microcrystalline wax. Alternatively, the wax may be a vegetable or animal wax (e.g. esters of C₁₄₋₃₂ fatty acids and C₁₄₋₃₂ fatty alcohols), such as beeswax or hydrogenated castor oil. In some preferred embodiments the viscosity-increasing ingredient is an inorganic substance such as fumed silica (e.g. available under the trade name Aerosil®, such as Aerosil® 200P, which is a high purity amorphous anhydrous colloidal silicon dioxide). The viscosity-increasing ingredient may also be selected from magnesium stearate, aluminium stearate, a sterol such as cholesterol, a long-chain saturated fatty alcohol such as cetostearyl alcohol. In some preferred embodiments the viscosity-increasing ingredient is a silicone rubber or wax, such as Dow Corning@ ST-Elastomer 10 or Dow Corning@ Silky Wax 10. Dow Corning@ ST-Elastomer 10 and/or Aerosil® are particularly preferred. The composition may include more than one viscosity-increasing ingredient, such as two or three viscosity-increasing ingredients. The viscosity-increasing ingredient may be a mixture of acrylamide acryloyldimethyl taurate copolymer, isohexadecane and polysorbate 80, such as that available under the trade name SEPINEO P600. The viscosity-increasing ingredient may be an alkylpolyglucoside, such as that available under the trade name SEPINEO SE68.

Also viscosity increasing compounds such as polysaccharides such as hyaluronic acid, chitosan, pectin, xanthan gum, agar, carrageenan, tragacanth, starch, polydextrose cellulose derivative such as HEC, HPC, HPMC, MC, polyacrylates, polycarbophyl, polyvinylalcohol, polyvinylpirrolydones can be used.

The composition may include one or more viscosity-increasing ingredient selected from the following group; polyacrylates, polycarbophils, poloxamers, hyaluronic acid, xanthan, natural polysaccharides, chitosan and cellulose-derivatives. Suitable cellulose-derivative emulsifiers include hydroxyalkyl cellulose polymers (e.g. hydroxymethyl cellulose, hydroxyethyl cellulose, hydroxypropyl cellulose (hypromellose) and hydroxypropylmethyl cellulose), carboxymethyl cellulose (Carmellose), methylhydroxyethyl cellulose and methylcellulose, carbomer (e.g. Carbopol®), carrageenans.

In certain preferred embodiments, the viscosity-increasing ingredient is hydroxyethyl cellulose, such as that available under the trade name Natrosol® (e.g. Natrosol® 250 HX, Natrosol® PLUS CS, Grade 300 etc.) and METHOCEL®. In certain preferred embodiments, the viscosity-increasing ingredient is hydroxypropyl cellulose, such as that available under the trade name Klucel® and METHOCEL®.

The amount of viscosity-increasing ingredient may vary (according to the viscosifying power of the ingredient), but the composition may include from about 0.5% to about 40% viscosity-increasing ingredient by weight of the composition. When the viscosity-increasing ingredient is microcrystalline wax it is typically present in an amount of from about 0.5% to about 10% by weight of the composition. Where the viscosity-increasing ingredient is SEPINEO P600, it is typically included in an amount of from about 1% to about 10% by weight of the composition, e.g. about 2.5% by weight of the composition. Where the viscosity-increasing ingredient is SEPINEO SE68, it is typically included in an amount of from about 2% to about 30% by weight of the composition, e.g. about 5% by weight of the composition. Where the viscosity-increasing ingredient is Dow Corning® ST-Elastomer 10 and/or Aerosil®, it is typically included in an amount of from about 1% to about 10% by weight of the composition, e.g. 1%, 2%, 5% or 10% by weight of the composition.

Co-Solvent

In some embodiments, the composition may include a co-solvent selected from the group consisting of lower alcohols, such as n-propanol, isopropanol, n-butanol, 2-butanol and benzyl alcohol, and diols such as glycerol, propylene glycol and hexylene glycol. This may be preferred where dispersion of the ingenol-3-angelate is problematic. These co-solvents may also act as a penetration enhancer aiding the penetration of the ingenol-3-angelate into the skin. Glycerol also act as a humectant. Further humectants may be hexylene glycol or pentylene glycol. Humectants may be present in the formulation with a concentration of 0.1%-98%, such as 0.5-75%.

Addition of a co-solvent may result in an improved physical stability of the composition. The composition may include more than one co-solvent, e.g. two or three co-solvents. For example, the composition may include benzyl alcohol and isopropanol. The co-solvent may be present in an amount of from present in an amount of from about 0.5% to about 40% by weight of the composition, such as from about 5% to about 30%, e.g. about 10%, about 15%, about 20%, about 25%, or about 30% by weight of the composition.

Penetration Enhancer

In some embodiments where the gel composition would otherwise have unsatisfactory penetration characteristics, it may be useful to include one or more penetration enhancers. Typical penetration enhancers include propylene carbonate, transcutol ((2-(2-ethoxyethoxy)ethanol), pyrrolidones such as N-methylpyrrolidone or N-hydroxyalkylpyrrolidone, azone, menthol, eucalyptol, nicotinamide, glycerol, mono-di- or polyglycols, ethylacetate or Eugenol. In an embodiment the penetration enhancer is an oil soluble ingredient such as isopropyl myristate and/or isopropanol.

In one embodiment, the composition includes a penetration enhancer in an amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

In one embodiment, the co-solvent (which may function as a penetration enhancer) and a further penetration enhancer are both present in a combined amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

Acidifying Compound

The composition of the invention may include an acidifying compound, for example where the stability of the gel composition would otherwise be unsatisfactory. An acidifying compound is a compound capable of providing a net overall acidic environment to the composition which means that the gel compositions are sufficiently acidic for the ingenol-3-angelate to remain stable at room temperature (25° C.) for extended periods, e.g. for 2 years. Generally, the acidifying compounds described herein are compounds which give a pH to the composition of less than about 4.5, such as less than 4 or less than 3.5.

The composition may include more than one acidifying compounds, for instance it may include two or three acidifying compounds. The acidifying compound may be present in an amount of from about which may be included in the composition in an amount of from about 0.5% to about 10% by weight of the composition, such as from about 5% to about 9% by weight of the composition. In some embodiments, the one or more non-aqueous carriers or the aqueous buffer solution may act as an acidifying compound. The acidifying compound may be fumed silica, which may be included in the composition in an amount of from about 3% to about 10% by weight of the composition, such as from about 5% to about 9% by weight of the composition. Alternatively, the acidifying compound may be a fatty acid such as oleic acid, lactic acid, linoleic acid, stearic acid, lauric acid, palmitic acid, capric acid, caprylic acid, pelargonic acid, adipic acid, sebacic acid or enanthic acid. The fatty acid is typically present in an amount of from about 0.5% to about 5% by weight of the composition.

Keratinolytic Agents

In some embodiments. the composition includes a keratinolytic agent, such as an α-hydroxy acid or β-hydroxy acid. The use of a keratinolytic agent may improve penetration of the active substance, meaning that compositions comprising a keratinolytic agent are particularly useful for treating hyperkeratotic actinic keratosis.

Suitable keratinolytic agents for use in the compositions of the invention include retinoids, adapalene, tars, shale oil, allantoin, aluminium oxide, azelaic acid, benzoyl peroxide, lactic acid, salicylic acid, alcali and alkali earth sulfide, monochloroacetic acid, urea, and resorcin. Particular retinoids that may be suitable include retinol, retinaldehyde, retinoic acid, isotretinoin, adapalinen and tazarotene. Further keratinolytic agents include ammonium glycolate, ammonium lactate, betaine salicylate, calcium lactate, calcium thioglycolate, glycolic acid, lactic acid, phenol, potassium lactate and sodium lactate.

In one embodiment, the composition includes an α-hydroxy acid selected from glycolic acid, lactic acid, mandelic acid, malic acid, citric acid and tartaric acid. In another embodiment, the composition includes a β-hydroxy acid such as salicylic acid. Preferably, the keratinolytic agent is salicylic acid.

The keratinolytic agent (e.g. α-hydroxy acid or β-hydroxy acid) may be present in an amount of from about 0.1% to about 20% by weight of the composition, e.g. about 0.5%, 1.0%, 2.5%, 5.0%, 7.5%, 10%, 15% or 20% by weight of the composition. Preferably, the composition includes salicylic acid in an amount of from about 0.1% to about 20% by weight of the composition, e.g. about 0.5%, 1.0%, 2.5%, 5.0%, 7.5%, 10%, 15% or 20% by weight of the composition.

Particularly Preferred Compositions

The inventors have found that certain compositions are particularly preferred.

In one embodiment, the composition comprises ingenol-3-angelate, benzyl alcohol, citric acid, sodium citrate, water, glycerol, macrogol stearyl, liquid paraffin and Aerosil® 200P. The composition may comprise ingenol-3-angelate in an amount of 0.05% by weight of the composition, benzyl alcohol in an amount of 9% by weight of the composition, citric acid in an amount of 0.14% by weight of the composition, sodium citrate in an amount of 0.035% by weight of the composition, water in an amount of 2.6% by weight of the composition, glycerol in an amount of 10% by weight of the composition, macrogol stearyl in an amount of 5% by weight of the composition, liquid paraffin in an amount of 76.275% by weight of the composition and Aerosil® 200P in an amount of 5% by weight of the composition.

In one embodiment, the compositions comprises Ingenol 3-angelate, Benzyl alcohol, Isopropyl Alcohol, 1% citrate buffer pH 2, Hydroxyethyl cellulose. In one embodiment, the compositions may comprise Ingenol 3-angelate in an amount of 0.5 mg/g, Benzyl alcohol in an amount of 9 mg/g, Isopropyl Alcohol in an amount of 150 mg/g-250 mg/g, 1% citrate buffer pH 2.8 in an amount of 725.5-815.5 mg/g, Hydroxyethyl cellulose HX (Natrosol® 250 HX) in an amount of 15 mg/g.

In an embodiment the composition comprises ingenol-3-angelate, benzyl alcohol, isopropyl alcohol, hydroxyethyl cellulose, citrate buffer and cyclomethicone or glycerol. In an embodiment the composition may comprise ingenol-3-angelate in an amount of 1 mg/g, benzyl alcohol in an amount of 9 mg/g, isopropyl alcohol in an amount of 300 mg/g, hydroxyethyl cellulose in an amount of 15 mg/g, and cyclomethicone in an amount of 50 mg/g or glycerol in an amount of 100-200 mg/g. The compositions may comprise citrate buffer.

In an embodiment the composition comprises ingenol-3-angelate, benzyl alcohol, Sepineo™ P600, propylene glycol, Polysorbate 80 and citrate buffer. In an embodiment the composition may comprise ingenol-3-angelate in an amount of 0.15%, Benzyl alcohol in an amount of 5 mg/g, Sepineo P600 in an amount of 25 mg/g, PG in an amount of 210 mg/g-764.5 mg/g, Polysorbate 80 in an amount of 5 mg/g, and Citrate buffer

In an embodiment the composition comprises ingenol-3-angelate, benzyl alcohol, isopropyl alcohol, buffer, Sepineo P600, and propylene glycol.

In an embodiment the composition comprises ingenol-3-angelate, ethyl alcohol, tween 80, Span 80, Citrate buffer and Sepineo P600.

In an embodiment the composition comprises ingenol-3-angelate, benzylalcohol, isopropyl alcohol, buffer, hydroxypropyl cellulose or hydroxyethyl cellulose, and propylene glycol and/or hexylene glycol and/or glycerol.

In an embodiment the composition may comprise ingenol-3-angelate in an amount of 0.5 mg/g, benzylalcohol in an amount of 9 mg/g, isopropyl alcohol in an amount of 250 mg/g, hydroxypropyl cellulose or hydroxyethyl cellulose in an amount of 20 mg/g-70 mg/g and propylene glycol and/or hexylene glycol and/or glycerol each in an amount of 100 mg/g.

In an embodiment the composition comprises ingenol-3-angelate, vitamin E, polyethylene glycol succinate, isopropanol, Sepineo P600, citrate buffer and benzyl alcohol.

In an embodiment the composition comprises ingenol-3-angelate, Nanosolve, Sepineo P600 citrate buffer and benzyl alcohol.

In an embodiment, the composition comprises ingenol-3-angelate, citric acid, water, PPO 10-15 stearylethyl, macrogolglycerol rincinoleate, and optionally carmellose sodium. In an embodiment, the composition may comprise ingenol-3-angelate in an amount of 0.05%, PPO 10-15 stearylethyl in an amount of 10%, macrogolglycerol rincinoleate in an amount of 20-35% and optionally carmellose sodium in an amount of 3%—all in weight of the composition

In an embodiment, the composition comprises PEP005, Citrate buffer pH 3.5, Polysorbate 80, Glycerol monocaprylocaprate (type I), Triglyceride, medium chain, Silica, colloidal anhydrous. In an embodiment, the composition comprises ingenol-3-angelate, citrate buffer pH 3.5, glycerol, Polyoxamer 407 and poloxamer 188. In an embodiment, the composition may comprise ingenol-3-angelate in an amount of 0.05%, glycerol in an amount of 10%, Polyoxamer 407 in an amount of 21.0-21.1% and poloxamer 188 in an amount of 3.0%-3.02% and water with citric acid, sodium citrate or citrate buffer.

In an embodiment the composition comprises ingenol-3-angelate, isopropyl myristate, lecithin and citrate buffer. In an embodiment, the composition comprises ingenol-3-angelate, benzyl alcohol, transcutol, citrate buffer pH 2.6, Seppineo P600 and propylene glycol.

In an embodiment the composition comprises cyclomethicone and/or glycerol. In an embodiment, the composition comprises glycerol.

Optional Disclaimers

In one embodiment, the composition does not consist essentially of:
(i) ingenol-3-angelate, medium chain triglycerides, polyoxyethylene-2-stearyl ether, benzyl alcohol, fumed silica, water buffered to pH 2.6-3.7 with citrate buffer and liquid paraffin; or
(ii) ingenol-3-angelate, isopropanol, polyoxyethylene-2-stearyl ether, benzyl alcohol, fumed silica, water buffered to pH 2.6-3.7 with citrate buffer and liquid paraffin.

In one embodiment, the composition does not consist essentially of:
(i) ingenol-3-angelate, benzyl alcohol, citric acid, citrate, water, glycerol, polyoxyethylene-2-stearyl ether, liquid paraffin and Aerosil 200P;
(ii) ingenol-3-angelate, benzyl alcohol, citric acid, citrate, water, glycerol, isopropanol, polyoxyethylene-2-stearyl ether, liquid paraffin and Aerosil 200P; or
(iii) ingenol-3-angelate, benzyl alcohol, citric acid, citrate, water, propylene glycol, isopropanol, polyoxyethylene-2-stearyl ether, liquid paraffin and Aerosil 200P.

In one embodiment, if the composition comprises ingenol-3-angelate in an amount of 0.05% by weight of the composition, then the composition does not consist essentially of:
(i) ingenol-3-angelate, benzyl alcohol, citric acid, citrate, water, glycerol, polyoxyethylene-2-stearyl ether, liquid paraffin and Aerosil 200P;
(ii) ingenol-3-angelate, benzyl alcohol, citric acid, citrate, water, glycerol, isopropanol, polyoxyethylene-2-stearyl ether, liquid paraffin and Aerosil 200P; or
(iii) ingenol-3-angelate, benzyl alcohol, citric acid, citrate, water, propylene glycol, isopropanol, polyoxyethylene-2-stearyl ether, liquid paraffin and Aerosil 200P.

In one embodiment, the composition does not consist essentially of:
(i) ingenol-3-angelate in an amount of 0.05% by weight of the composition, benzyl alcohol in an amount of 0.9% by weight of the composition, citric acid in an amount of 0.14% by weight of the composition, citrate in an amount of 0.035% by weight of the composition, water in an amount of 2.6% by weight of the composition, glycerol in an amount of 10% by weight of the composition, polyoxyethylene-2-stearyl ether in an amount of 5% by weight of the composition, liquid paraffin in an amount of 76.275% by weight of the composition and Aerosil 200P in an amount of 5% by weight of the composition;
(ii) ingenol-3-angelate in an amount of 0.05% by weight of the composition, benzyl alcohol in an amount of 0.9% by weight of the composition, citric acid in an amount of 0.14% by weight of the composition, citrate in an amount of 0.035% by weight of the composition, water in an amount of 2.6% by weight of the composition, glycerol in an amount of 10% by weight of the composition, isopropanol in an amount of 10% by weight of the composition, polyoxyethylene-2-stearyl ether in an amount of 5% by weight of the composition, liquid paraffin in an amount of 66.275% by weight of the composition and Aerosil 200P in an amount of 5% by weight of the composition; or
(iii) ingenol-3-angelate in an amount of 0.05% by weight of the composition, benzyl alcohol in an amount of 0.9% by weight of the composition, citric acid in an amount of 0.14% by weight of the composition, citrate in an amount of 0.035% by weight of the composition, water in an amount of 2.6% by weight of the composition, propylene glycol in an amount of 10% by weight of the composition, isopropanol in an amount of 10% by weight of the composition, polyoxyethylene-2-stearyl ether in an amount of 5% by weight of the composition, liquid paraffin in an amount of 66.275% by weight of the composition and Aerosil 200P in an amount of 5% by weight of the composition.

In an embodiment, the aqueous topical gel composition comprising ingenol-3-angelate, wherein:
(iv) the ingenol-3-angelate is not present in the composition in an amount of 0.02% by weight of the composition, or from 0.08% to 0.1% by weight of the composition; and
(v) if the composition consists essentially of ingenol-3-angelate in an amount of 0.05% by weight of the composition, isopropyl alcohol in an amount of 30% by weight of the composition, hydroxyethyl cellulose in an amount of 1.5% of the composition, benzyl alcohol in an amount of 0.9% by weight of the composition and citrate buffer in an amount of 67.55% by weight of the composition, then the pH of the citrate buffer is other than 2.8;
(vi) if the composition consists essentially of ingenol-3-angelate in an amount of 0.015% by weight of the composition, isopropyl alcohol in an amount of 30% by weight of the composition, hydroxyethyl cellulose in an amount of 1.5% of the composition, benzyl alcohol in an amount of 0.90% by weight of the composition and citrate buffer in an amount of 67.585% by weight of the composition, then the pH of the citrate buffer is other than 2.8.

Stability of the Compositions

The inventors have found that compositions of the invention can exhibit very favorable stability properties.

In some embodiments, the composition is chemically stable, where chemically stable (or chemical stability) means that less than 10% of the ingenol-3-angelate degrades when the gel is stored for 2 years at 25° C. In some preferred embodiments, less than 6% of the ingenol-3-angelate degrades over a storage period of 2 years. An approximation of chemical stability can be obtained by subjecting the composition to stability studies at 25° C. for 6 months: if less than about 2.5% of the ingenol-3-angelate has degraded after 6 months at 25° C. then a shelf-life of 2 years at room temperature is expected, i.e. less than 10% of the ingenol-3-angelate will be expected to degrade over a storage period of 2 years at 25° C. An approximation of chemical stability at room temperature can also be obtained by subjecting the composition to accelerated stability studies at 40° C. for 3 months. If less than about 2.5% of the substance, e.g. ingenol-3-angelate, has degraded after 3 months at 40° C., a shelf-life of 2 years at room temperature is considered to be feasible. These studies are carried out according to ICH Humidity Guidelines, at conditions of 25° C.±2°, 60% RH±5% and/or 40° C.±2°, 75% RH±5%, in hermetically sealed containers.

Preferred chemically stable gels include, after storage for 2 years at 25° C., less than 5% by weight of total ingenanes in the composition are ‘A’ and/or ‘B’. Thus, if the total amount of ‘A’ and ‘B’ exceeds 5% by weight of the total ingenanes, the gel's shelf-life is not ideal. An approximation of the amount of ‘A’ and/or ‘B’ in the embodiments can be carried out in the same manner as described for ingenol-3-angelate above.

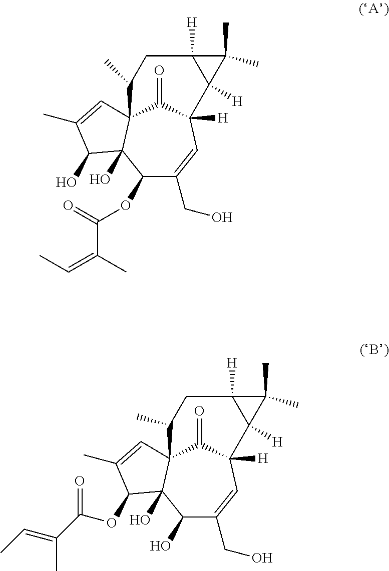

In some embodiments, where the composition does not fulfill the above criteria for chemical stable, the composition does exhibit a markedly improved stability over prior art hydrogel (Picato®). In particular these compositions preferably include gels where ingenol-3-angelate does not degrade by more than 15% when the gel is stored for 2 years at room temperature and/or the gels contain less than 12% of ‘A’ and/or ‘B’ by weight of total ingenanes. These values can be approximated as described above for the chemical stable gels.

In some embodiments, the composition is physically stable, where physically stable (or physical stability) means that the composition retains its macroscopic and microscopic appearance over the shelf-life of the product, e.g. any dissolved ingenol-3-angelate does not precipitate from the solvent phase.

In some embodiments, the composition is chemically stable and physically stable.

The inventors have found that a number of the compositions of the invention exhibit very favorable stability properties.

Penetration and Permeation of the Compositions

Compositions of the invention can exhibit very favorable skin penetration characteristics. Skin penetration means the flux of the active ingredient into the different layers of the skin, i.e. the stratum corneum, epidermis and dermis, after application of the gel to the skin.

In some embodiments, the compositions exhibit greater flux, according to the in vitro diffusion test, of ingenol-3-angelate into the stratum corneum, epidermis and dermis after application of the gel to skin than does a reference gel of ingenol-3-angelate; wherein the reference gel (a) has the same strength of ingenol-3-angelate as the topical gel composition, (b) consists essentially of ingenol-3-angelate, benzyl alcohol, isopropyl alcohol in an amount of 30% by weight of the formulation, hydroxyethyl cellulose in an amount of 1.5% by weight of the formulation and citrate buffer solution in an amount of 67.55% by weight of the formulation, and (c) is prepared by mixing ingenol-3-angelate with benzyl alcohol, and then adding the remaining components to the mixture of ingenol-3-angelate and benzyl alcohol in the order of: isopropyl alcohol, a citrate buffer solution formed from citric acid in an amount of 0.56% by weight of the formulation, sodium citrate dihydrate in an amount of 0.14% by weight of the formulation and water in an amount of 66.85% by weight of the formulation, and then hydroxyethyl cellulose to form the reference gel.

The in vitro diffusion test is performed as described in the examples section below.

If the total amount of ingenol-3-angelate in the stratum corneum, epidermis and dermis as a percentage of the applied dose, as determined in step (h), is higher than for the reference gel (e.g. PICATO® at the same strength of ingenol-3-angelate as the topical gel composition), then the composition is said to exhibit more penetration (i.e. greater flux of the active ingredient into the stratum corneum, epidermis and dermis after application of the gel to the skin).

In some embodiments, the composition exhibits less penetration than the reference gel according to this assay, i.e. the total amount of ingenol-3-angelate in the stratum corneum, epidermis and dermis (combined) as a percentage of the applied dose, as determined in step (h), is lower than for the reference gel (e.g. PICATO® at the same strength of ingenol-3-angelate as the topical gel composition).

Skin permeation means the flux of the active ingredient through the skin into the systemic circulation or, in case of in vitro studies, the receptor fluid of the Franz cell apparatus used in the experiment, after application of the gel to the skin. In some embodiments, the composition exhibits less permeation than does the reference gel according to this assay, where less potent permeation means that the amount of ingenol-3-angelate in the receptor fluid as a percentage of the applied dose, as determined in step (h), is lower than for the reference gel (e.g. PICATO® at the same strength of ingenol-3-angelate as the topical gel composition). This may be desirable to avoid unnecessary levels of systemic ingenol-3-angelate.

Medical Treatments and Uses

The invention also provides a method for treating a dermal disease or condition, comprising topical administration of a gel of the invention to a mammal. Topical administration means that the compositions are applied cutaneously i.e. to the external skin on the body.

The invention also provides a gel of the invention for use in treating a dermal disease or condition.

The invention also provides the use of ingenol-3-angelate and a non-aqueous carrier in the manufacture of a gel medicament for treating a dermal disease or condition.

The uses and methods are useful for the topical treatment of dermal diseases or conditions including actinic keratosis, seborrheic keratosis, skin cancer, warts, keloids, scars, photoaged or photodamaged skin, and acne. In particular, the uses and methods are particularly useful for the topical treatment of actinic keratosis. The uses and methods may, for instance, be useful for the topical treatment of hyperkeratotic actinic keratosis.

The uses and methods may be used for the topical treatment of skin cancers such as non-melanoma skin cancer, malignant melanoma, Merkel cell carcinoma, squamous cell carcinoma or basal cell carcinoma (including superficial basal cell carcinoma and nodular basal cell carcinoma).

The uses and methods may be used for the topical treatment of warts, e.g. human papilloma virus (HPV) infections on the skin, genitals and mouth.

The uses and methods may be used for the topical treatment of photodamaged skin such as fine lines, wrinkles and UV-ageing. UV-ageing is often manifested by an increase in the epidermal thickness or epidermal atrophy, most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting in a leathery appearance, skin fragility and irregular pigmentation, loss of tone and elasticity, as well as wrinkling, dryness, sunspots and deep furrow formation.

The uses and methods may be useful for reducing or minimizing scar tissue or improving cosmesis or functional outcome in a wound. For instance, the uses and methods may be useful for improving functional outcome in a wound which is cutaneous, chronic or diabetes associated, e.g. when the wound includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant, immune response conditions, oral wounds, stomach or intestinal wounds, damaged cartilage or bone, amputation sides and corneal lesions.

Therefore, in some embodiments, the uses and methods are cosmetic.

Typically, the uses and methods are lesion specific, i.e. they are focused on a lesion being treated and do not extend to any larger degree to the surrounding skin. In other embodiments, however, the uses and methods can extend to a larger area than the lesions, and this can usefully lead to treatment of emerging lesions or sub-surface pre-lesions. Also, it can be convenient to apply a gel to an area which includes several lesions, rather than applying it to each individual lesion in that area. The lesions could be of any size (i.e. surface area), e.g. greater than about 30 000 mm², greater than about 20 000 mm², greater than about 10 000 mm², greater than about 5000 mm², greater than about 1000 mm², greater than about 500 mm², greater than about 250 mm², or greater than about 150 mm² Typically, the lesion size is about 30 000 mm², about 20 000 mm², about 10 000 mm², about 5000 mm², about 1000 mm², about 500 mm², about 250 mm², about 150 mm², about 100 mm², about 75 mm², about 50 mm², about 25 mm² or about 10 mm².

In the treatment of, for example, actinic keratosis on the face and/or scalp of a subject, a gel composition of the invention may be applied on the face and scalp to the affected skin area (treatment area) once a day for 3 consecutive days. In the treatment of, for example, actinic keratosis on the trunk and/or extremities of a subject, a gel composition of the invention may be applied on the trunk and extremities to the affected skin area (treatment area) once a day for 2 consecutive days Immediately following application of a gel to the treatment area, subjects should wash their hands.

The gel compositions of the invention are typically packaged in hermetically sealed containers, e.g. a unit dose tube. A unit dose tube would typically contain about 0.5 g of gel. Preferably, one unit dose tube (tube with screw cap or individual packets) may be used for one treatment area.

### DEFINITIONS

The term “aqueous” means that the content of free water in the composition is greater than or equal to about 2% by weight, preferably more than about 5% by weight of the composition, e.g. more than about 10%, 20%, 30%, 40%, 50%, 60%, 70%, 80% or 90% by weight of the composition.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

### MODES FOR CARRYING OUT THE INVENTION

The invention is further illustrated by the followings examples. It will be appreciated that the examples are for illustrative purposes only and are not intended to limit the invention as described above. Modification of detail may be made without departing from the scope of the invention.

### Example A

### Preparation of Compositions of the Invention

The following compositions were prepared:

Composition Series 8

15A

PEP005 0.5 mg/g

Benzyl Alcohol 5 mg/g

Citrate buffer pH 2.3 600 mg/g

Propylene glycol 59.5 mg/g

Sepineo P600 35 mg/g

Isopropyl alcohol 300 mg/g

Composition Series 14

02A

PEP005 0.5 mg/g

Lipoid SPC 100 mg/g

Isopropanol 100 mg/g

Sepineo P600 25 mg/g

Citrate buffer pH 3.0 764.5 mg/g

Benzyl alcohol 10 mg/g

06A

PEP005 0.5 mg/g

Vitamin E polyethylene glycol succinate 100 mg/g

Isopropanol 100 mg/g

Sepineo P600 35 mg/g

Citrate buffer pH 3.0 754.5 mg/g

Benzyl alcohol 10 mg/g

08A

PEP005 0.5 mg/g

NanoSolve 100 mg/g

Sepineo P600 30 mg/g

Citrate buffer pH 3.0 859.5 mg/g

Benzyl alcohol 10 mg/g

Composition Series 20

01A

PEP005 0.5 mg/g

Nanosolve 100 mg/g

Hydroxyethyl cellulose HX (Natrosol® 250 HX) 15 mg/g

Citrate buffer 874.5 mg/g

Benzyl alcohol 10 mg/g

Composition Series 33

01A

PEP005 0.5 mg/g

Benzyl alcohol 9 mg/g

Isopropyl Alcohol 300 mg/g

1% citrate buffer pH 2.8 675.5 mg/g

Hydroxyethyl cellulose HX (Natrosol® 250 HX) 15 mg/g

02A

PEP005 0.5 mg/g

Benzyl alcohol 9 mg/g

Isopropyl Alcohol 250 mg/g

1% citrate buffer pH 2.8 725.5 mg/g

Hydroxyethyl cellulose HX (Natrosol® 250 HX) 15 mg/g

03A

PEP005 0.5 mg/g

Benzyl alcohol 9 mg/g

Isopropyl Alcohol 200 mg/g

1% citrate buffer pH 2.8 775.5 mg/g

Hydroxyethyl cellulose HX (Natrosol® 250 HX) 15 mg/g

04A

PEP005 0.5 mg/g

Benzyl alcohol 9 mg/g

Isopropyl Alcohol 150 mg/g

1% citrate buffer pH 2.8 825.5 mg/g

Hydroxyethyl cellulose HX (Natrosol® 250 HX) 15 mg/g

05A

PEP005 0.5 mg/g

Benzyl alcohol 9 mg/g

Isopropyl Alcohol 100 mg/g

1% citrate buffer pH 2.8 875.5 mg/g

Hydroxyethyl cellulose HX (Natrosol® 250 HX) 15 mg/g

06A

PEP005 0.5 mg/g

Benzyl alcohol 20 mg/g

1% citrate buffer pH 2.8 965.5 mg/g

Hydroxyethyl cellulose HX (Natrosol® 250 HX) 15 mg/g

07A

PEP005 0.5 mg/g

Benzyl alcohol 20 mg/g

Isopropyl Alcohol 100 mg/g

1% citrate buffer pH 2.8 865.5 mg/g

Hydroxyethyl cellulose HX (Natrosol® 250 HX) 15 mg/g

08A

PEP005 0.5 mg/g

Benzyl alcohol 20 mg/g

Isopropyl Alcohol 150 mg/g

1% citrate buffer pH 2.8 815.5 mg/g

Hydroxyethyl cellulose HX (Natrosol® 250 HX) 15 mg/g

09A

PEP005 0.5 mg/g

Benzyl alcohol 20 mg/g

Isopropyl Alcohol 200 mg/g

1% citrate buffer pH 2.8 765.5 mg/g

Hydroxyethyl cellulose HX (Natrosol® 250 HX) 15 mg/g

Composition Series 34

01A

PEP005 0.50 mg/g

Benzyl alcohol 9 mg/g

Isopropyl Alcohol 300 mg/g

1% citrate buffer stock pH 2.8 675.5 mg/g

Hydroxyethyl cellulose HX (Natrosol® 250 HX) 15 mg/g

02A

PEP005 0.5 mg/g

Benzyl alcohol 9 mg/g

Isopropyl Alcohol 300 mg/g

2.5% citrate buffer stock pH 2.8 675.5 mg/g

Hydroxyethyl cellulose HX (Natrosol® 250 HX) 15 mg/g

03A

PEP005 0.5 mg/g

Benzyl alcohol 9 mg/g

Isopropyl Alcohol 300 mg/g

5% citrate buffer stock pH 2.8 675.5 mg/g

Hydroxyethyl cellulose HX (Natrosol® 250 HX) 15 mg/g

04A

PEP005 0.5 mg/g

Benzyl alcohol 9 mg/g

Isopropyl Alcohol 300 mg/g

7.5% citrate buffer stock pH 2.8 675.5 mg/g

Hydroxyethyl cellulose HX (Natrosol® 250 HX) 15 mg/g

05A

PEP005 0.5 mg/g

Benzyl alcohol 9 mg/g

Isopropyl Alcohol 300 mg/g

10% citrate buffer stock pH 2.8 675.5 mg/g

Hydroxyethyl cellulose HX (Natrosol® 250 HX) 15 mg/g

06A

PEP005 0.5 mg/g

Benzyl alcohol 9 mg/g

Isopropyl Alcohol 300 mg/g

1% citrate buffer stock pH 2.8 670.5 mg/g

Hydroxyethyl cellulose HX (Natrosol® 250 HX) 20 mg/g

07A

PEP005 0.5 mg/g

Benzyl alcohol 9 mg/g

Isopropyl Alcohol 300 mg/g

1% citrate buffer stock pH 2.8 665.5 mg/g

Hydroxyethyl cellulose HX (Natrosol® 250 HX) 25 mg/g

08A

PEP005 0.5 mg/g

Benzyl alcohol 9 mg/g

Isopropyl Alcohol 300 mg/g

1% citrate buffer stock pH 2.8 665.5 mg/g

Hydroxyethyl cellulose HX (Natrosol® 250 HX) 30 mg/g

09A

PEP005 0.5 mg/g

Benzyl alcohol 9 mg/g

Isopropyl Alcohol 300 mg/g

1% citrate buffer stock pH 2.8 655.5 mg/g

Hydroxyethyl cellulose HX (Natrosol® 250 HX) 40 mg/g

Composition Series 35

01A

PEP005 0.5 mg/g

Benzyl alcohol 9 mg/g

Isopropyl Alcohol 300 mg/g

1% citrate buffer stock pH 2.8 675.5 mg/g

HEC Natrosol 250 HX (Natrosol® 250 HX) 15 mg/g

18A

PEP005 0.5 mg/g

Benzyl alcohol 9 mg/g

Isopropyl Alcohol 300 mg/g

1% citrate buffer stock pH 2.8 640.5 mg/g

Aerosil 972P 50 mg/g

09A

PEP005 0.5 mg/g

Benzyl alcohol 9 mg/g

Isopropyl Alcohol 300 mg/g

1% citrate buffer stock pH 2.8 660.5 mg/g

Natrosol 30 mg/g

11A

PEP005 0.5 mg/g

Benzyl alcohol 9 mg/g

Isopropyl Alcohol 300 mg/g

1% citrate buffer stock pH 2.8 640.5 mg/g

Klucel JF Ph 50 mg/g

12A

PEP005 0.5 mg/g

Benzyl alcohol 9 mg/g

Isopropyl Alcohol 300 mg/g

1% citrate buffer stock pH 2.8 620.5 mg/g

Klucel JF Ph 70 mg/g

14A

PEP005 0.5 mg/g

Benzyl alcohol 9 mg/g

Isopropyl Alcohol 300 mg/g

1% citrate buffer stock pH 2.8 580.5 mg/g

Klucel JF Ph 110 mg/g

19A

PEP005 0.5 mg/g

Benzyl alcohol 9 mg/g

Isopropyl Alcohol 300 mg/g

1% citrate buffer stock pH 2.8 565.5 mg/g

Klucel LF Ph 125 mg/g

Composition Series 36

05A

PEP005 0.5 mg/g

Benzyl alcohol 9 mg/g

Isopropyl Alcohol 300 mg/g

1% citrate buffer stock pH 2.8 590.5 mg/g

Hypromellose 15 cps 100 mg/g

06A

PEP005 0.5 mg/g

Benzyl alcohol 9 mg/g

Isopropyl Alcohol 300 mg/g

1% citrate buffer stock pH 2.8 570.5 mg/g

Hypromellose 15 cps 120 mg/g

09A

PEP005 0.5 mg/g

Benzyl alcohol 9 mg/g

Isopropyl Alcohol 300 mg/g

1% citrate buffer stock pH 2.8 530.5 mg/g

Hypromellose 160 cps

10A

PEP005 0.5 mg/g

Benzyl alcohol 9 mg/g

Isopropyl Alcohol 300 mg/g

1% citrate buffer stock pH 2.8 660.5 mg/g

Propylene glycol alginate 30 mg/g

16A

PEP005 0.5 mg/g

Benzyl alcohol 9 mg/g

Isopropyl Alcohol 300 mg/g

1% citrate buffer stock pH 2.8 600.5 mg/g

Methocel E50 90 mg/g (methylcellulose and hydroxypropyl methylcellulose)

Composition Series 37

05A

PEP005 0.5 mg/g

Benzyl alcohol 9 mg/g

Isopropyl Alcohol 300 mg/g

Ethanol 100 mg/g

Water 475.5 mg/g

Salicylic acid 100 mg/g

Hydroxyethyl cellulose HX (Natrosol® 250 HX) 15 mg/g

06A

PEP005 0.5 mg/g

Benzyl alcohol 9 mg/g

Isopropyl Alcohol 300 mg/g

Ethanol 100 mg/g

Water 525.5 mg/g

Salicylic acid 50 mg/g

Hydroxyethyl cellulose HX (Natrosol® 250 HX) 15 mg/g

07A

PEP005 0.5 mg/g

Benzyl alcohol 9 mg/g

Isopropyl Alcohol 300 mg/g

Ethanol 100 mg/g

Water 555.5 mg/g

Salicylic acid 20 mg/g

Hydroxyethyl cellulose HX (Natrosol® 250 HX) 15 mg/g

09A

PEP005 0.5 mg/g

Benzyl alcohol 9 mg/g

Isopropyl Alcohol 300 mg/g

Ethanol 100 mg/g

1% citrate buffer pH 2.8 575.5 mg/g

Hydroxyethyl cellulose HX (Natrosol® 250 HX) 15 mg/g

Composition Series 39

01A

PEP005 0.5 mg/g

Benzyl alcohol 9 mg/g

Isoporopyl Alcohol 300 mg/g

1% citrate buffer pH 2.8 675.5 mg/g

HEC HX 15 mg/g

11A

PEP005 0.5 mg/g

Benzyl alcohol 9 mg/g

Isoporopyl Alcohol 300 mg/g

1% citrate buffer pH 2.8 640.5 mg/g

Seppineo P600 50 mg/g

13A

PEP005 0.5 mg/g

Benzyl alcohol 9 mg/g

Isoporopyl Alcohol 300 mg/g

1% citrate buffer pH 2.8 590.5 mg/g

Kollidon 90FP 100 mg/g

Composition Series 41

01A

PEP005 0.5 mg/g

Benzyl alcohol 9 mg/g

Isopropyl Alcohol 300 mg/g

1% citrate buffer pH 2.5 675.5 mg/g

HEC (hydroxyethyl cellulose) HX 15 mg/g

06A

PEP005 0.5 mg/g

Benzyl alcohol 9 mg/g

Isopropyl Alcohol 300 mg/g

1% citrate buffer pH 2.5 590.5 mg/g

Polyvinyl acetate E530 100 mg/g

11A

PEP005 0.5 mg/g

Benzyl alcohol 9 mg/g

Isopropyl Alcohol 300 mg/g

1% citrate buffer pH 2.5 680.5 mg/g

HEC (hydroxyethyl cellulose) HHX 10 mg/g

Composition Series 42

04A

PEP005 0.5 mg/g

Benzyl alcohol 9 mg/g

Isopropyl Alcohol 300 mg/g

1% citrate buffer pH 2.5 660.5 mg/g

SepiMax 30 mg/g (polyacrylate)

07A

PEP005 0.5 mg/g

Benzyl alcohol 9 mg/g

Isopropyl Alcohol 300 mg/g

1% citrate buffer pH 2.5 675.5 mg/g

Xanthan gum 15 mg/g

11A

PEP005 0.5 mg/g

Benzyl alcohol 9 mg/g

Isopropyl Alcohol 300 mg/g

1% citrate buffer pH 2.5 675.5 mg/g

Xanthan gum 15 mg/g

Composition Series 47

01A

PEP005 0.5 mg/g

Benzyl alcohol 9 mg/g

Isopropyl Alcohol 300 mg/g

1.5% buffer pH 2.5 670.5

Hydroxyethyl cellulose HX 20 mg/g

02A

PEP005 0.5 mg/g

Benzyl alcohol 9 mg/g

Isopropyl Alcohol 300 mg/g

1.5% buffer pH 2.6 670.5 mg/g

Hydroxyethyl cellulose HX 20 mg/g

03A

PEP005 0.5 mg/g

Benzyl alcohol 9 mg/g

Isopropyl Alcohol 300 mg/g

1.5% buffer pH 2.7 670.5 mg/g

Hydroxyethyl cellulose HX 20 mg/g

04A

PEP005 0.5 mg/g

Benzyl alcohol 9 mg/g

Isopropyl Alcohol 300 mg/g

1.5% buffer pH 2.8 670.5 mg/g

Hydroxyethyl cellulose HX 20 mg/g

05A

PEP005 0.5 mg/g

Benzyl alcohol 9 mg/g

Isopropyl Alcohol 250 mg/g

1.5% buffer pH 2.8 720.5 mg/g

Hydroxyethyl cellulose HX 20 mg/g

06A

PEP005 0.5 mg/g

Benzyl alcohol 9 mg/g

Isopropyl Alcohol 250 mg/g

1.5% buffer pH 2.7 720.5 mg/g

Hydroxyethyl cellulose HX 20 mg/g

07A

PEP005 0.5 mg/g

Benzyl alcohol 9 mg/g

Isopropyl Alcohol 250 mg/g

1.5% buffer pH 2.7 720.5 mg/g

Hydroxyethyl cellulose HX 20 mg/g

08A

PEP005 0.5 mg/g

Benzyl alcohol 9 mg/g

Isopropyl Alcohol 250 mg/g

1.5% buffer pH 2.8 720.5 mg/g

Hydroxyethyl cellulose HX 20 mg/g

Composition Series 48

01A

PEP005 0.5 mg/g

Benzyl alcohol 9 mg/g

Isopropyl Alcohol 250 mg/g

1.5% buffer pH 2.6 690.5 mg/g

Natrosol+330 50 mg/g

02A

PEP005 0.5 mg/g

Benzyl alcohol 9 mg/g

Isopropyl Alcohol 250 mg/g

1.5% buffer pH 2.6 690.5 mg/g

Klucel GF Ph 50 mg/g

03A

PEP005 0.5 mg/g

Benzyl alcohol 9 mg/g

Isopropyl Alcohol 250 mg/g

1.5% buffer pH 2.5 615.5 mg/g

Klucel LF Ph 125 mg/g

04A

PEP005 0.5 mg/g

Benzyl alcohol 9

Isopropyl Alcohol 250 mg/g

1.5% buffer pH 2.5 mg/g

Methocel E50 90 mg/g (methylcellulose and hydroxypropyl methylcellulose)

05A

PEP005 0.5 mg/g

Benzyl alcohol 9 mg/g

Isopropyl Alcohol 250 mg/g

1.5% buffer pH 2.5 690.5 mg/g

SeppineoP600 50 mg/g

06A

PEP005 0.5 mg/g

Benzyl alcohol 9 mg/g

Isopropyl Alcohol 250 mg/g

1.5% buffer pH 2.5 710.5 mg/g

SepiMax 30 mg/g (polyacrylate)

07A

PEP005 0.5 mg/g

Benzyl alcohol 9 mg/g

Isopropyl Alcohol 250 mg/g

1.5% buffer pH 2.5 725.5 mg/g

Xanthan gum 15 mg/g

08A

PEP005 0.5 mg/g

Benzyl alcohol 9 mg/g

Isopropyl Alcohol 250 mg/g

3% buffer pH 2.5 725.5 mg/g

Xanthan gum 15 mg/g

Composition Series 53

01A

PEP005 0.5 mg/g

Benzyl alcohol 9 mg/g

Isopropyl Alcohol 250 mg/g

1.5% buffer pH 2.6 570.5 mg/g

Klucel GF Ph 70 mg/g

Glycerol 100 mg/g

13A

PEP005 0.5 mg/g

Benzyl alcohol 9 mg/g

Isopropyl Alcohol 250 mg/g

1.5% buffer pH 2.6 670.5 mg/g

Klucel GF Ph 70 mg/g

14A

PEP005 0.5 mg/g

Benzyl alcohol 9 mg/g

Isopropyl Alcohol 250 mg/g

1.5% buffer pH 2.6 470.5 mg/g

Klucel GF Ph 70 mg/g

Glycerol 100 mg/g

Propylene glycol 100 mg/g

15A

PEP005 0.5 mg/g

Benzyl alcohol 9 mg/g

Isopropyl Alcohol 250 mg/g

1.5% buffer pH 2.6 470.5 mg/g

Klucel GF Ph 70 mg/g

Glycerol 100 mg/g

Hexylene glycol 100 mg/g

16A

PEP005 0.5 mg/g

Benzyl alcohol 9 mg/g

Isopropyl Alcohol 250 mg/g

1.5% buffer pH 2.6 490.5 mg/g

Natrosol+330 CS 50 mg/g

Glycerol 100 mg/g

Hexylene glycol 100 mg/g

17A

PEP005 0.5 mg/g

Benzyl alcohol 9 mg/g

Isopropyl Alcohol 250 mg/g

1.5% buffer pH 2.6 520.5 mg/g

Hydroxyethyl cellulose HX (Natrosol® 250 HX) 20 mg/g

Glycerol 100 mg/g

Hexylene glycol 100 mg/g

18A

PEP005 0.5 mg/g

Benzyl alcohol 9 mg/g

Isopropyl Alcohol 250 mg/g

1.5% buffer pH 2.6 490.5 mg/g

Natrosol+330 CS 50 mg/g

Glycerol 100 mg/g

Propylene glycol 100 mg/g

19A

PEP005 0.5 mg/g

Benzyl alcohol 9 mg/g

Isopropyl Alcohol 250 mg/g

1.5% buffer pH 2.6 520.5 mg/g

Hydroxyethyl cellulose HX (Natrosol® 250 HX) 20 mg/g

Glycerol 100 mg/g

Propylene glycol 100 mg/g

20A

PEP005 0.5 mg/g

Benzyl alcohol 9 mg/g

Isopropyl Alcohol 200 mg/g

1.5% buffer pH 2.6 420.5 mg/g

Klucel GF Ph 70 mg/g

Glycerol 100 mg/g

Propylene glycol 100 mg/g

Hexylene glycol 100 mg/g

21A

PEP005 0.5 mg/g

Benzyl alcohol 9 mg/g

Isopropyl Alcohol 200 mg/g

1.5% buffer pH 2.6 440.5 mg/g

Natrosol+330 CS 50 mg/g

Glycerol 100 mg/g

Propylene glycol 100 mg/g

Hexylene glycol 100 mg/g

22A

PEP005 0.5 mg/g

Benzyl alcohol 9 mg/g

Isopropyl Alcohol 200 mg/g

1.5% buffer pH 2.6 470.5 mg/g

Hydroxyethyl cellulose HX (Natrosol® 250 HX) 20 mg/g

Glycerol 100 mg/g

Propylene glycol 100 mg/g

Hexylene glycol 100 mg/g

Composition Series 55

01A

PEP005 0.5 mg/g

Benzyl alcohol 9 mg/g

1.5% citrate buffer pH 2.5 910.5 mg/g

Natrosol+330 CS 30 mg/g

CremophorEL 50 mg/g

02A

PEP005 0.5 mg/g

Benzyl alcohol 9 mg/g

1.5% citrate buffer pH 2.5 810.5 mg/g

Natrosol+330 CS 30 mg/g

CremophorEL 50 mg/g

Glycerol 85% 100 mg/g

03A

PEP005 0.5 mg/g

Benzyl alcohol 9 mg/g

1.5% citrate buffer pH 2.5 810.5 mg/g

Natrosol+330 CS 30 mg/g

CremophorEL 50 mg/g

Propylene glycol 100 mg/g

04A

PEP005 0.5 mg/g

Benzyl alcohol 9 mg/g

Isopropyl Alcohol 100 mg/g

1.5% citrate buffer pH 2.5 810.5 mg/g

Natrosol+330 CS 30 mg/g

CremophorEL 50 mg/g

05A

PEP005 0.5 mg/g

Benzyl alcohol 9 mg/g

Isopropyl Alcohol 200 mg/g

1.5% citrate buffer pH 2.5 710.5 mg/g

Natrosol+330 CS 30 mg/g

CremophorEL 50 mg/g

06A

PEP005 0.5 mg/g

Benzyl alcohol 9 mg/g

1.5% citrate buffer pH 2.5 910.5 mg/g

Natrosol+330 CS 30 mg/g

Polysorbate 80 50 mg/g

07A

PEP005 0.5 mg/g

Benzyl alcohol 9 mg/g

1.5% citrate buffer pH 2.5 810.5 mg/g

Natrosol+330 CS 30 mg/g

Glycerol 85% 100 mg/g

Polysorbate 80 50 mg/g

08A

PEP005 0.5 mg/g

Benzyl alcohol 9 mg/g

1.5% citrate buffer pH 2.5 810.5 mg/g

Natrosol+330 CS 30 mg/g

Polysorbate 80 50 mg/g

Propylene glycol 100 mg/g

09A

PEP005 0.5 mg/g

Benzyl alcohol 9 mg/g

Isopropyl Alcohol 100 mg/g

1.5% citrate buffer pH 2.5 810.5 mg/g

Natrosol+330 CS 30 mg/g

Polysorbate 80 50 mg/g

10A

PEP005 0.5 mg/g

Benzyl alcohol 9 mg/g

Isopropyl Alcohol 200 mg/g

1.5% citrate buffer pH 2.5 710.5 mg/g

Natrosol+330 CS 30 mg/g

Polysorbate 80 50 mg/g

Composition Series 58

01A

PEP005 0.5 mg/g

Benzyl alcohol 9 mg/g

Isopropyl Alcohol 250 mg/g

1.5% buffer pH 2.6 390.5 mg/g

SeppineoP600 50 mg/g

Propylene glycol 300 mg/g

02A

PEP005 0.5 mg/g

Benzyl alcohol 9 mg/g

Isopropyl Alcohol 200 mg/g

1.5% buffer pH 2.6 440.5 mg/g

SeppineoP600 50 mg/g

Propylene glycol 300 mg/g

03A

PEP005 0.5 mg/g

Benzyl alcohol 9 mg/g

Isopropyl Alcohol 150 mg/g

1.5% buffer pH 2.6 490.5 mg/g

SeppineoP600 50 mg/g

Propylene glycol 300 mg/g

04A

PEP005 0.5 mg/g

Benzyl alcohol 9 mg/g

Isopropyl Alcohol 100 mg/g

1.5% buffer pH 2.6 540.5 mg/g

SeppineoP600 50 mg/g

Propylene glycol 300 mg/g

05A

PEP005 0.5 mg/g

Benzyl alcohol 9 mg/g

Isopropyl Alcohol 50 mg/g

1.5% buffer pH 2.6 590.5 mg/g

SeppineoP600 50 mg/g

Propylene glycol 300 mg/g

Composition Series 59

01A

PEP005 0.5 mg/g

Benzyl alcohol 9 mg/g

Transcutol 300 mg/g (2-(2-ethoxyethoxy)ethanol)

1.5% buffer pH 2.6 340.5 mg/g

SeppineoP600 50 mg/g

Propylene glycol 300 mg/g

02A

PEP005 0.5 mg/g

Benzyl alcohol 9 mg/g

Transcutol 250 mg/g (2-(2-ethoxyethoxy)ethanol)

1.5% buffer pH 2.6 390.5 mg/g

SeppineoP600 50 mg/g

Propylene glycol 300 mg/g

03A

PEP005 0.5 mg/g

Benzyl alcohol 9 mg/g

Transcutol 200 mg/g (2-(2-ethoxyethoxy)ethanol)

1.5% buffer pH 2.6 440.5 mg/g

SeppineoP600 50 mg/g

Propylene glycol 300 mg/g

04A

PEP005 0.5 mg/g

Benzyl alcohol 9 mg/g

Transcutol 150 mg/g (2-(2-ethoxyethoxy)ethanol)

1.5% buffer pH 2.6 490.5 mg/g

SeppineoP600 50 mg/g

Propylene glycol 300 mg/g

05A

PEP005 0.5 mg/g

Benzyl alcohol 9 mg/g

Transcutol 100 mg/g (2-(2-ethoxyethoxy)ethanol)

1.5% buffer pH 2.6 540.5 mg/g

SeppineoP600 50 mg/g

Propylene glycol 300 mg/g

06A

PEP005 0.5 mg/g

Benzyl alcohol 9 mg/g

Transcutol 50 mg/g (2-(2-ethoxyethoxy)ethanol)

1.5% buffer pH 2.6 590.5 mg/g

SeppineoP600 50 mg/g

Propylene glycol 300 mg/g

Composition Series 60

03A

PEP005 0.5 mg/g

Benzyl alcohol 9 mg/g

Isopropyl alcohol 300 mg/g

Buffer 637.5 mg/g

SeppineoP600 50 mg/g

Composition Series G (Amounts in % (m/m)

1(A)

PEP005 0.05%

Citrate buffer pH 3.5 54.95%

PPO 10-15 Stearylether 10.0%

Macrogolglycerol ricinoleate (PEG 35 Castor oil) 35.0%

Formulation 1, Self-Thickened Macrogolglycerol Ricinoleate Gel (˜pH 4.5)

The active substance was first dissolved in the oil. Thereafter, the emulsifier was added and mixed with the oil to a homogeneous solution. Finally, the aqueous phase was added in small portions. Between the added portions, the formulation was mixed with a spatula to a homogeneous, viscous gel.

2(A)

PEP005 0.05%

Citrate buffer pH 5.0 54.95%

PPO 10-15 Stearylether 10.0%

Macrogolglycerol ricinoleate (PEG 35 Castor oil) 35.0%

Formulation 2, Self-Thickened Macrogolglycerol Ricinoleate Gel (˜pH 5.6)

This formulation was manufactured in the same way as Formulation 1.

3(A)

Citric acid, anhydrous 0.5%

Water 66.45%

PEP005 0.05%

PPO 10-15 Stearylether 10.0%

Macrogolglycerol ricinoleate (PEG 35 Castor oil) 20.0%

Carmellose sodium 3.0%

Formulation 3, Carmellose Sodium Thickened Macrogolglycerol Ricinoleate Gel (˜pH 4.4)

The active substance was first dissolved in the oil. Thereafter, the emulsifier was added to the oil and mixed to a homogeneous solution. The polymer was added to the aqueous phase. After gelling of the aqueous phase, the oil phase was added to the viscous aqueous phase.

9(A)

Citric acid, anhydrous 0.47%

Water 59.38%

Sodium citrate 0.1%

PEP005 0.05%

PPO 10-15 Stearylether 10.0%

Macrogolglycerol ricinoleate (PEG 35 Castor oil) 30.0%

Carmellose sodium 3.0%

Formulation 9, Self-Thickened Macrogolglycerol Ricinoleate Gel (˜pH 3.5)

This formulation was manufactured in the same way as Formulation 1.

4(A)

Citrate buffer pH 3.5 65.78%

Glycerol 10.05%

PEP005 0.05%

Poloxamer 407 21.1%

Poloxamer 188 3.02%

Formulation 4, Self-Thickened Poloxamer Gel (˜pH 4.2)

First water and glycerol were mixed and cooled on an ice bath. Thereafter, small portions of the poloxamers were added during stirring. The dispersion was mixed until a clear homogeneous solution was obtained. Finally, the active substance was added and dissolved during stirring and the temperature of the formulation was adjusted to room temperature.

10(A)

Citric acid, anhydrous 0.42%

Sodium citrate 0.08%

Glycerol 10.00%

PEP005 0.05%

Poloxamer 407 21.0%

Poloxamer 188 3.00%

Water 65.45%

Formulation 10, Self-Thickened Poloxamer Gel (˜pH 3.5)

This formulation was manufactured in the same way as Formulation 4.

7(A)

Citrate buffer pH 3.5 70.85%

Glycerol 4.00%

PEP005 0.05%

Triglyceride, medium chain 10.0%

Macrogol oleyl ether (oleth-20) 8.00%

Macrogol oleyl ether (oleth-5) 6.00%

PEG-150 distearate 1.10%

Formulation 7, PEG-150 Distearate Thickened Macrogol Oleyl Ether Gel (˜pH 3.9)

The active substance was first dissolved in the oil. Thereafter, the emulsifiers were added. The aqueous phase was mixed and both the aqueous and the oil phases were heated to 77° C. Small portions of water phase were added to the oil phase. The solution was equilibrated prior to addition of the next portion. For the first portions the solution remained turbid whereas after further additions the solution became clear. After the inflection point when the solution lost viscosity and started to foam, larger portions of aqueous phase were added. After about 1 h all of the aqueous phase had been added and the nanoemulsion was cooled in a cold water bath. The thickener was thereafter added. Mixing with a propeller stirrer was required to solubilize the thickener.

8(A)

Citrate buffer pH 3.5 73.85%

PEP005 0.05%

Triglyceride, medium chain 9.0%

Paraffin liquid 1.00%

Lecithin 1.00%

Polysorbate 80 10.0%

PEG-150 distearate 1.10%

Formulation 8, PEG-150 Distearate Thickened Polysorbate 80/Lecithin Gel (˜pH 5.6)

The active substance was first dissolved in the medium chain triglyceride and liquid paraffin oils. Thereafter, first the emulsifiers (lecithin and polysorbate 80) and then the water phase were added and the mixture was stirred to a white/orange homogeneous emulsion. The emulsion was heated to 60° C. and high pressure homogenized (150 MPa) during 36 minutes (corresponding to 8 cycles) prior to cooling to room temperature. Finally, the thickener was added. Mixing with a propeller stirrer was required to solubilize the thickener.

6(A)

Citrate buffer pH 3.5 3.00%

PEP005 0.05%

Isopropyl myristate 51.95%

Lecithin 45.0%

Formulation 6, Self-Thickened Lecithin Gel

First lecithin was dissolved in the oil at 50° C. Then the aqueous phase was added and a viscous, clear solution was obtained. Finally, the active substance was added and the solution was stirred during 2 h at about 45° C.

5(A)

Citrate buffer pH 3.5 21.15%

Polysorbate 80 35.0%

PEP005 0.05%

Glycerol monocaprylocaprate (type I) 9.0%

Triglyceride, medium chain 26.5%

Silica, colloidal anhydrous 8.30%

Formulation 5, Silica Thickened Polysorbate 80 Gel

The active substance was first dissolved in the medium chain triglyceride and glycerol monocaprylocaprate oils. A slightly turbid solution was obtained. Then the emulsifier was added to the oil phase and the phase was mixed to a homogeneous, clear solution. The aqueous phase was thereafter added slowly during stirring to the oil/emulsifier phase. Finally, silica was added in portions to the solution with stirring in between each addition.

Chemical Stability Evaluation:

The following compositions are predicted to have improved stability over Picato® at room temperature (25° C.), but may not reach 2 year stability at 25° C.:

1A, 3A, 4A, 5A, 9A, 10A

Reference Example (i)—Composition Series 18

01A

PEP005 0.5 mg/g

Labrasol® 470 mg/g

Plurol® Oleique 120 mg/g

Caprylic/capric triglyceride 250 mg/g

Buffer 149.5 mg/g

Benzyl alcohol 10 mg/g

02A

PEP005 0.5 mg/g

Labrasol® 380 mg/g

Plurol® Oleique 100 mg/g

Caprylic/capric triglyceride 75 mg/g

Buffer 434.5 mg/g

Benzyl alcohol 10 mg/g

05A

PEP005 0.5 mg/g

Labrasol® 630 mg/g

Labrafil® M1944 50 mg/g

Transcutol® 100 mg/g (2-(2-ethoxyethoxy)ethanol)

Capryol® 105 mg/g

Buffer 99.5 mg/g

Benzyl alcohol 10 mg/g

06A

PEP005 0.5 mg/g

Labrasol® 180 mg/g

Plurol® Oleique 180 mg/g

Isostearyl isostearate 250 mg/g

Buffer 379.5 mg/g

Benzyl alcohol 10 mg/g

Reference Example (ii)—Composition Series 27

10A

PEP005 0.5 mg/g

Benzyl Alcohol 9 mg/g

Water 670.5 mg/g

Hyaluronic acid 20 mg/g

Isopropyl alcohol 300 mg/g

11A

PEP005 0.5 mg/g

Benzyl Alcohol 9 mg/g

Water 670.5 mg/g

Isopropyl alcohol 300 mg/g

Propylene glycol alginate 20 mg/g

These reference example compositions (Examples (i) and (ii)) are not gel compositions, but provide examples of compositions that are compatible with PEP005. These non-gel compositions could be converted into gels by the addition of a suitable amount of gelling agent.

Composition Series 62 (Niosomes)

03A

PEP005 0.5 mg/g

Ethyl alcohol 160 mg/g

Tween 80 50 mg/g

Span 80 50 mg/g

Citrate buffer ad 1000

Sepineo P600 35 mg/g

04A

PEP005 0.5 mg/g

Ethyl alcohol 160 mg/g

Tween 80 50 mg/g

Span 80 50 mg/g

Citrate buffer ad 1000

Hydroxyethyl cellulose 15 mg/g

Niosome Preparation

The buffer is prepared. Divide the buffer into two parts. The lipid surfactants and API are dissolved in the alcohol. One part of the buffer and the alcoholic solution are added under constant mixing. The dispersion is homogenised by high pressure homogenisation.

Final Formulation

The other part of buffer is used for the hydrogel preparation.

Disperse the gelling agent in the buffer.

When homogeneous hydrogel is obtained add the Niosome dispersion under homogenization.

Composition Series 68

01A (Control Used in Example E—Cream Formulation)

PEP005 (0.015%),

MCT Miglyol 812 (55.45%)

Benzyl Alcohol (0.5%),

SepineoP600 (2.5%),

Propylene Glycol (21%),

polysorbate 80 (0.5%),

citrate buffer pH 2.9 (20.035%)

02A

PEP005 0.15%

Benzyl alcohol 5 mg/g

Sepineo P600 25 mg/g

PG 764.5 mg/g

Polysorbate 80 5 mg/g

Citrate buffer 200.35 mg/g

16A

PEP005 0.15%

Benzyl alcohol 5 mg/g

Sepineo P600 25 mg/g

PG 210 mg/g

Polysorbate 80 5 mg/g

Citrate buffer 754.85 mg/g

18A

PEP005 0.15%

Benzyl alcohol 5 mg/g

Natrosol+330 CS 30 mg/g

PG 210 mg/g

Citrate buffer 754.85 mg/g

Preparation of Formulations of Composition Series 68

API Stock:

Weigh an appropriate amount of PEP005 into red cap flask containing the desired amount of benzyl alcohol. Stir until dissolution.

Buffer (1% w/w):

weight out appropriate amount of Citric acid and Na-Citrate and water. Adjust pH to 2.9±0.2 with either Citric acid or NaOH of e.g. 0.5-1M. Store at 2-8° C. Water phase: In a suitable container mix the buffer, Propylene Glycol, polysorbate 80 and Sepineo P600 (Sepineo P600: remember to shake bottle well before weighing). Mix or Homogenize and let to swell. If HEC is used instead of Sepineo, solubilize HEC in the buffer and stir/mix until homogeneity. Afterwards, add the water soluble components followed by mixing.

Final Formulation:

Add API stock to the water phase under homogenisation (at low rate). When all oil phase is incorporated in the water phase, homogenise with Silverson for about 15 minute 5000 rpm at room temperature.

Storage: at 2-8° C.

Composition Series 72 (Niosomes)

05A (Brij 05 Niosome)

PEP005 0.5 mg/g

Benzylalcohol 10 mg/g

Isopropyl alcohol 50 mg/g

Brij 05 100 mg/g (Macrogol Oleyl Ether)

0.2% Citrate buffer pH 3 ad 1000

07A (Span 83/EO Niosome 6/4)

PEP005 0.5 mg/g

Benzylalcohol 10 mg/g

Isopropyl alcohol 160 mg/g

Sorbitansesquioleate 60 mg/g

Ethyloleate 40 mg/g

0.2% Citrate buffer pH 3 ad 1000

Sepineo P600 35 mg/g

08A (Cithrol PG3PR/EO Niosome 6/4)

PEP005 0.5 mg/g

Benzylalcohol 10 mg/g

Isopropyl alcohol 100 mg/g

Cithrol PG3PR 60 mg/g

Ethyloleate 40 mg/g

0.2% Citrate buffer pH 3 ad 1000

Sepineo P600 35 mg/g

Niosome Preparation

The buffer is prepared. Divide the buffer into two parts (600 mg/g and the rest). The lipid surfactants and are dissolved in the alcohol, and then the benzylalcohol with or without API is added and mixed until dissolution. One part of the buffer (600 mg/g) is added slowly to the alcoholic solution under constant mixing (Silversson 3000-4500 rpm). Afterwards the dispersion is homogenised by high pressure homogenisation 3×5 min 500-800 Bar.

Final Formulation

The rest of buffer is added to the gelling agent.

Disperse the gelling agent in the buffer by Silverson applying low shear rate.

Then add the Niosome dispersion under further homogenization.

Adjust pH to 3.5.

Composition Series 73

07A

PEP005 1 mg/g

Benzyl alcohol 9 mg/g

Isopropyl alcohol 300 mg/g

HEC Natrosol HX 15 mg/g

Citrate buffer pH 2,8 675 mg/g

08A (Hydroalcoholic Gel)

PEP005 1 mg/g

Benzyl alcohol 9 mg/g

Isopropyl alcohol 300 mg/g

Hydroxyethyl cellulose Natrosol HX 15 mg/g

Citrate buffer 625 mg/g

Cyclomethicone 50 mg/g

10A

PEP005 1 mg/g

Benzyl alcohol 9 mg/g

Isopropyl alcohol 300 mg/g

Hydroxyethyl cellulose Natrosol HX 15 mg/g

Citrate buffer 575 mg/g

Glycerol 85% 100 mg/g

11A

PEP005 1 mg/g

Benzyl alcohol 9 mg/g

Isopropyl alcohol 300 mg/g

Hydroxyethyl cellulose Natrosol HX 15 mg/g

Citrate buffer 474 mg/g

Glycerol 85% 200 mg/g

Preparation of Composition Series 73
Buffer stock solution: Weight out water, Citric acid monohydrate and sodium citrate and mix. Check and set pH to 2.8 with either Citric acid or NaOH solutions
API stock solution: Charge appropriate amount of API in a small glass bottle and solubilize it in Benzyl alcohol and IPA.
HEC stock solution: In an appropriate container, charge the desired amount of citrate buffer. Under homogenisation, add slowly the HEC powder. Use silverson at about 2000 rpm and increase to about 5000 rpm. Mix until homogeneity (no lumps).
If neither glycerol/IPM/cyclomethicone are existed, mix API stock solution with HEC stock solution by whisk until homogeneity.
If IPM/cyclomethicone, they should be first mixed with the API stock solution until homogeneity.
If Glycerol is existed in the formulation, it should be first mixed with HEC stock solution.
Storage: 2-8° C.

Composition Series 80

06A

PEP005 0.5 mg/g

Benzyl alcohol 9 mg/g

DC Silmogen carrier 100 mg/g

Isopropylalcohol 200 mg/g

Sepineo P600 25 mg/g

Glycerol 85% 15 mg/g

Citrate buffer pH 2.8 ad 1000

08A

PEP005 0.5 mg/g

Benzyl alcohol 9 mg/g

DC Silmogen carrier 20 mg/g

Isopropylalcohol 200 mg/g

Sepineo P600 25 mg/g

Glycerol 85% 15 mg/g

Citrate buffer pH 2.8 ad 1000

12A

PEP005 0.5 mg/g

Benzyl alcohol 9 mg/g

DC ST-Elastomer 10 100 mg/g

Ethanol 200 mg/g

Sepineo P600 25 mg/g

Glycerol 85% 15 mg/g

Citrate buffer pH 2.8 ad 1000

Preparation of Composition Series 80 (06A, 08A and 12A)

Buffer stock: Weight out appropriate amount of Citric acid to make a stock solution, e.g. 0.5-1.0 L. Set pH with NaOH of e.g. 0.5-1M. Buffer stock solution should be stored at 2-8° C.

API stock phase: Charge appropriate amount of API in small glass bottle with screw cap (preventing alcohol evaporation). Add appropriate amount of Benzyl Alcohol.

Sepineo gel phase: In suitable beaker (for silicones) add Buffer stock.

In suitable container (for silicones) mix remaining Buffer stock solution, and Sepineo P600 (shake bottle well before weighing).

Mix or Homogenize and let to swell.

Add appropriate amount of glycerol

Final Formulation

Weigh the appropriate amount silicones (DC ST-Elastomer 10, DC Dimethiconol Blend 20, Cyclomethicone, Silmogen carrier), mix them

Take the homogeneous hydrogel phase which has the appropriate weight for the final formulation.

Add Glycerol and the alcohol (Ethanol, isopropanol) by manual mixing

Place the homogenizer (allowed to silicones) into it.

Pour very slowly (adding little fraction at a time) the silicon phase into the hydrogel under high shear.

Add the benzyl alcohol or API stock phase under further homogenization

Fill 3.5 g formulation in 5 g plastic laminate tubes.

### Example B

A number of compositions of the invention were tested for chemical stability. This testing required extraction of ingenol-3-angelate from the composition by dissolution in a solvent mixture of acetonitrile and phosphoric acid. Following extraction, organic impurities were identified using reversed phase HPLC with UV detection at 220 nm. The following composition from Example A was found to be stable after 6 months at 25° C., indicating that less than 10% of the ingenol-3-angelate would be expected to degrade over a storage period of 2 years at room temperature (25° C.) and/or that the formulations were expected to contain less than 5% by weight of total ingenanes of the degradation products ‘A’ and/or ‘B’:
Composition 62, formulation 03A.
Composition series G, formulation 6A
Compositions series 68 formulations 02A and 16A

The following compositions were found to have a markedly improved stability over prior art hydrogel (Picato®), indicating ingenol-3-angelate was not expected to degrade by more than 15% when the gel is stored for 2 years at room temperature and/or the gels were expected to contain less than 12% of ‘A’ and/or ‘B’ by weight of total ingenanes:
Composition 14, formulation 06A and 08A
Composition 33, formulation 08A
Composition 53, formulation 14A, 15A, 17A, 20A and 21A
Composition 58, formulation 01A
Composition 59, formulation 01A
Composition 62, formulation 03A
Composition 68, formulation 02A
Composition series G, formulations 1A, 3A, 4A, 5A, 9A, 10A

### Example C

### Skin Penetration and Permeation Studies

To investigate the skin penetration and permeation of ingenol-3-angelate from compositions of the invention, an in vitro skin diffusion test was conducted.

Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Flow-through Franz-type diffusion cells with an available diffusion area of 3.14 cm² and receptor volumes ranging from 11.1 to 12.6 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After half an hour the saline was replaced by receptor medium, 0.04 M isotonic phosphate buffer, pH 7.4 (35° C.), containing 4% bovine serum albumin and left for hydration another hour. The inlet and outlet ports of the receptor chamber were connected to stainless steel HPLC tubing. The cells were connected to a 12-channel peristaltic pump, and the receptor fluid was pumped continuously through each cell and collected in vials placed at a fraction collector. A controller was used to program independently the duration of each fraction. Sink conditions were maintained at all times during the period of the study, i.e. the concentration of the active compounds in the receptor medium was below 10% of the solubility of the compounds in the medium.

The in vitro skin penetration and permeation was tested in 6 replicates (i.e. n=6). Each test composition was applied to the skin membrane at 0 hours in an intended dose of 4 mg/cm². A glass spatula was used for the application, and the residual amount of the composition was determined so as to give the amount of the composition actually applied on the skin.

Permeation and Penetration

The skin penetration and permeation experiment was allowed to proceed for 21 hours. Samples were then collected from the following compartments:

About 6 ml of the receptor fluid was sampled from each cell every third hour until 21 hours post application. The sample collection of the first 45 minutes was discarded due to the lag time of the system. The recipient fluid remaining in the diffusion cell at the end of the study corresponded to the 21 hour sample.

The stratum corneum was collected by tape stripping 10 times using D-Squame® tape (diameter 22 mm, CuDerm Corp., Dallas, Tex., USA). Each tape strip was applied to the test area using a standard pressure for 5 seconds and removed from the test area in one gentle, continuous move.

For each repeated strip, the direction of tearing off was varied. The viable epidermis and dermis was then sampled from the skin by taking a full biopsy of 3.14 cm² of the applied area for analysis. The skin surrounding the test area was discarded.

The concentration of ingenol-3-angelate in the samples was determined by LC-MS/MS.

Results

These studies allowed the amount of ingenol-3-angelate found in the stratum corneum, epidermis and dermis and receptor fluid to be calculated, as a percentage of the applied dose.

The following compositions from Example A exhibited more penetration than PICATO® at the same strength of ingenol-3-angelate by weight of the composition:
Composition series 33, formulations 02A and 09A

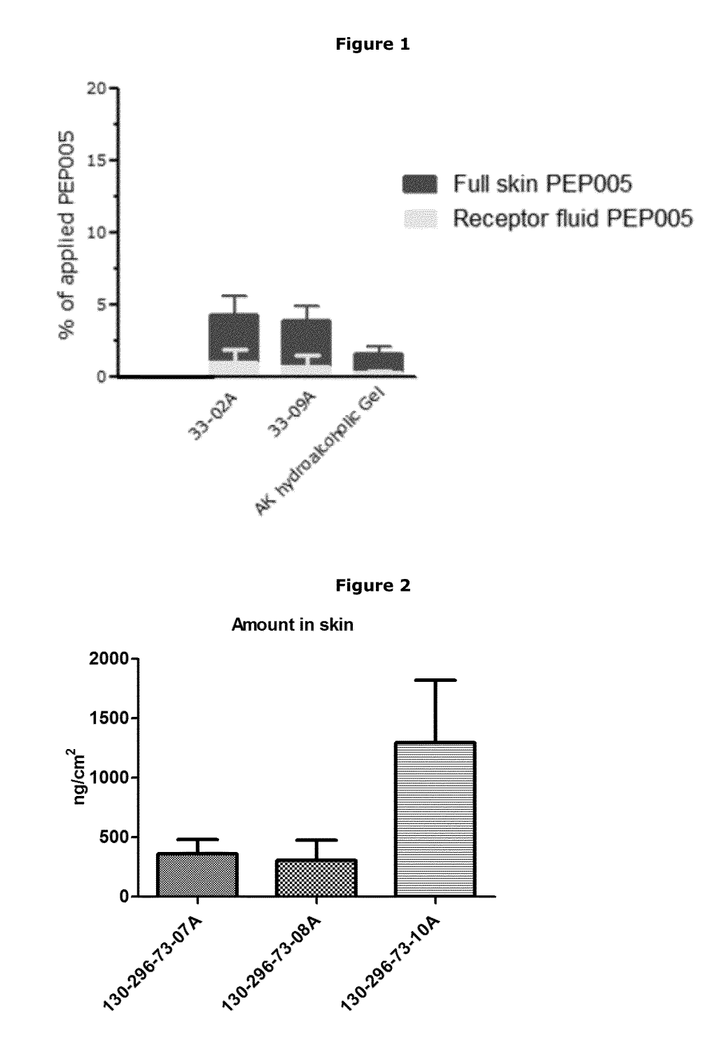

The data for composition series 33, formulations 02A and 09A are shown in FIG. 1, which show that the amount of ingenol-3-angelate found in the stratum corneum, epidermis and dermis after application of these compositions is significantly higher than the amount found in the stratum corneum, epidermis and dermis after application of PICATO® at the same strength of ingenol-3-angelate by weight of the composition.

In FIG. 2, The penetration performance of Composition 73-07 (reference), 73-08A and 10A on the ingenol-3-angelate content in the skin is illustrated. The effects show that addition of cyclomethicone compared to the reference example shows equal penetration. Addition of cyclomethicone provides a composition which has more favourable cosmetic properties which often provides improved patient compliance. In this case, it has been achieved without compromising the penetration into the skin.

Addition of glycerol in the compositions provide good results in this assay as also shown in the B16 model below.

### Example D

### Anti-Tumor Efficacy in B16 Melanoma Mice Model of Compositions

In this model B16 melanoma cells are injected intradermally in syngeneic C57BL/6 mice. After 3 days the mice are treated topically at the tumor bearing site once daily for two consecutive days with the test formulation. Tumor growth is monitored for 40 days and tumor size exceeding 250 mm3 or ulcerating tumor is used as termination point. Kaplan-Meyer survival curves is used to calculate efficacy of the test compound/formulation. This model is used as a surrogate for efficacy.

Result

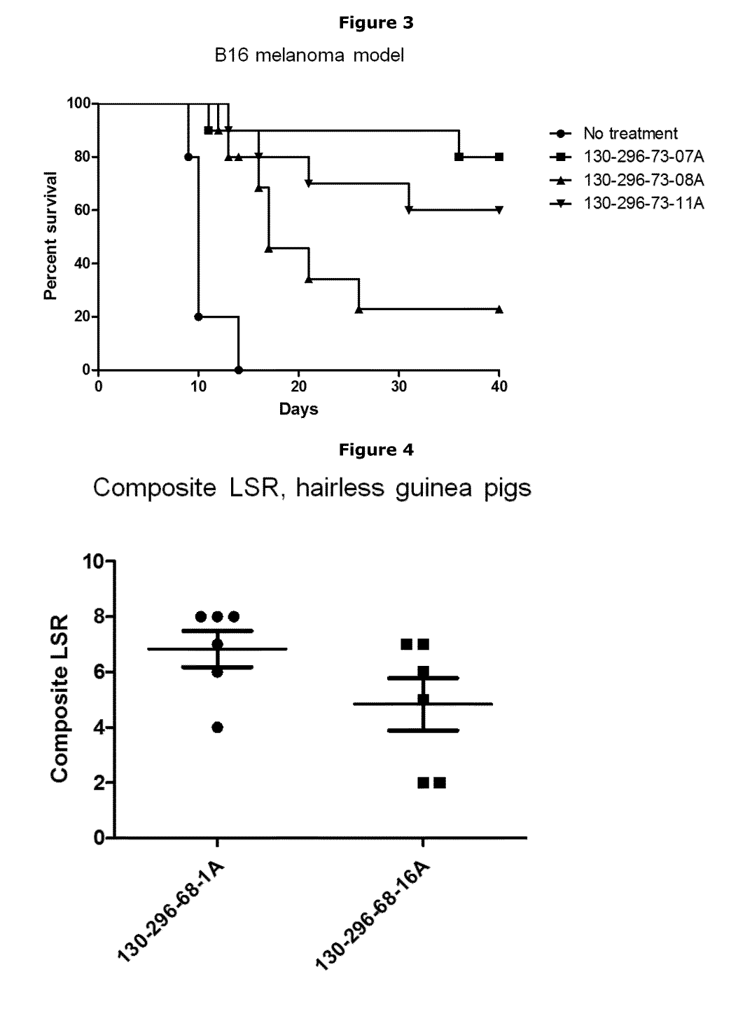

In FIG. 3, composition 73, formulation 07A (reference) 08A and 11A are evaluated in respect to anti-tumor efficacy performance.

Effect are shown for hydrogels containing cyclomethicone or glycerol potentially indicating that formulations containing cyclomethicone and glycerol are beneficial in anti-tumor efficacy.

### Example E

### Local Skin Reaction (LSR) Model in Hairless Guinea Pigs in Compositions

In this model hairless guinea pigs are treated topically with the test compounds on dorsal skin on a field of 1-2 cm2 in size. Subsequently, erythema, oedema, wounding and vascular leakage (intracutaneous bleeding) is assessed visually and score on a 0 to 3 scale, where 0 means normal and 3 is the most severe reaction, typically extending outside the treatment area for eythema and oedema, or covering the entire treatment area for wounding and vascular leakage. The composite LSR score is calculated as the sum of the four individual parameters. Thus, the composite LSR has a range from 0 to 12. Typically, the composite LSR peaks 24 h-48 h after administration of ingenol derivatives. The composite LSR is used as a surrogate endpoint for efficacy, assuming that higher potency of the formulation will lead to higher efficacy

Result

In FIG. 4, composition 68, formulation 01A and 16A are evaluated in respect to composite LSR on hairless guinea pig. Composition 68-01A is an oil-in-water cream containing approximately 50% MCT (medium chain triglycerides). Composition 68-16A is a hydrogel with the same composition as 68-01A but without the MCT. The hydrogel gave less LSR.

It will be understood that the invention has been described by way of example only and modifications may be made whilst remaining within the scope and spirit of the invention.

1. An aqueous topical gel composition comprising ingenol-3-angelate as a suspension.

2. The composition of claim 1, including ingenol-3-angelate as a crystalline suspension.

3. The composition of claim 1, wherein the total amount of ingenol-3-angelate in the composition is either 0.015% by weight or 0.05% by weight.

4.-18. (canceled)

19. The composition of claim 1, wherein the ingenol-3-angelate is present in an amount of about 0.0005%, 0.001%, 0.0025%, 0.005%, 0.01%, 0.015%, 0.025%, 0.05%, 0.075%, 0.1%, 0.125%, 0.15%, 0.2%, 0.25% or 0.5% by weight of the composition.

20. (canceled)

21. The composition of claim 1, wherein the composition is acidic.

22. The composition of claim 21, wherein the composition has a pH of less than about 4.5.

23. The composition of claim 1, wherein the composition includes an aqueous buffer solution.

24. (canceled)

25. (canceled)

26. The composition of claim 1, wherein the composition includes one or more emulsifiers.

27. (canceled)

28. The composition of claim 1, wherein the composition includes a non-aqueous carrier.

29. (canceled)

30. The composition of claim 1, wherein the composition includes a viscosity increasing ingredient.

31. (canceled)

32. The composition of claim 1, wherein the composition includes a co-solvent.

33. (canceled)

34. The composition of claim 1, wherein the composition includes a penetration enhancer.

35. (canceled)

36. The composition of claim 1, wherein the composition includes an acidifying compound.

37. (canceled)

38. The composition of claim 1, wherein the composition includes a hydrophilic nonionic surfactant.

39. (canceled)

40. The composition of claim 1, wherein the composition includes a lipophilic non-ionic surfactant.

41. (canceled)

42. The composition of claim 1, wherein the composition includes a hydrophilic nonionic surfactant and a lipophilic non-ionic surfactant.

43. The composition of claim 1, wherein the composition includes a silicone.

44. (canceled)

45. The composition of claim 1, wherein the composition includes a keratinolytic agent.

46. (canceled)

47. (canceled)

48. The composition of claim 1, wherein the composition is chemically stable.

49. The composition of claim 1, wherein the composition is physically stable.

50. The composition of claim 1, wherein the composition exhibits more penetration than a reference gel of the same strength of ingenol-3-angelate, according to an in vitro diffusion test.

51. (canceled)

52. The composition of claim 1, wherein the composition exhibits less permeation than a reference gel of the same strength of ingenol-3-angelate, according to an in vitro diffusion test.

53. The composition of claim 1, wherein the composition shows effect in the B16 melanoma model compared to the reference composition.

54. (canceled)

55. The composition of claim 1, wherein the composition show improved effect in the B16 melanoma model compared to the reference composition.

56. (canceled)

57. The composition of claim 1,
wherein the composition is a w/o micro emulsion gel.

58.-60. (canceled)

61. A method for treating a dermal disease or condition, comprising topically administering a composition of claim 1 to a mammal in need thereof.

62. The method of claim 61, wherein the dermal disease or condition is actinic keratosis.
